(19) United States
(12) Patent Application Publication
(10) Pub. No.: US 2011/0218189 A1
(43) Pub. Date: Sep. 8, 2011

(54) PYRROLO[2,3-d]PYRIMIDIN-2-YL-AMINE DERIVATIVES AS PKC-THETA INHIBITORS

(75) Inventors: Andrew Laird Roughton, Plainsboro, NJ (US); Koc-kan Ho, West Windsor, NJ (US); Michael Ohlmeyer, Plainsboro, NJ (US); David Diller, East Windsor, NJ (US); Irina Neagu, Plainsboro, NJ (US); Celia Kingsbury, Cream Ridge, NJ (US); Jui-Hsiang Chan, West Windsor, NJ (US); Johannes Petrus, Mmaria Lommerse, Oss (NL); Neeltje Miranda Teerhuis, Oss (NL); Jacobus Comelis Henricus Maria Wijkmans, Oss (NL); Ralf Plate, Oss (NL)

(73) Assignee: N.V. ORGANON, OSS (NL)

(21) Appl. No.: 12/936,604

(22) PCT Filed: Apr. 8, 2009
(86) PCT No.: PCT/EP09/54209

§ 371 Date: May 23, 2011

(30) Foreign Application Priority Data
Apr. 9, 2008 (US) 61/043593

(51) Int. Cl. A61K 31/55; C07D 487/04; A61K 31/519; A61K 31/5377; A61K 31/541; A61P 29/00; A61P 43/00

(52) U.S. Cl. 514/217.06; 544/280; 544/117; 544/61; 540/600; 514/265.1; 514/234.2; 514/228.5

(57) ABSTRACT

The present invention relates to a pyrrolo[2,3-d]pyrimidin-2-yl-amine derivative according to formula (I) wherein the variables are defined as in the specification, or to a pharmaceutically acceptable salt or solvate thereof. The present invention also relates to a pharmaceutical composition comprising one or more of said pyrrolo[2,3-d]pyrimidine-2-ylamine derivatives and to their use in therapy, for instance in the treatment of PKCθ mediated disorders.

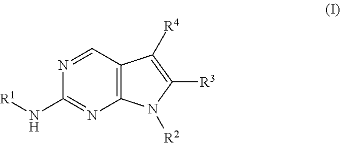

The present invention relates to pyrrolo[2,3-d]pyrimidin-2-yl-amine derivatives, to pharmaceutical compositions comprising these compounds and to their use in therapy, in particular to their use in the treatment of PKC-theta (PKCθ) mediated disorders.

Members of the protein kinase C (PKC) family of serine/threonine kinases play critical roles in the regulation of cellular differentiation and proliferation of diverse cell types. Ten mammalian members of PKC family have been identified and designated α, β, γ, δ, ε, ζ, η, θ, μ, and λ. The structure of PKCθ displays the highest homology with members of the Ca²⁺ independent novel PKC subfamily, including PKCδ, ε, and η. PKCθ is most highly related to PKCδ.

PKCθ is expressed predominantly in lymphoid tissue and skeletal muscle. It has been shown that PKCθ is essential for TCR-mediated T-cell activation but inessential during TCR-dependent thymocyte development. PKCθ, but not other PKC isoforms, translocates to the site of cell contact between antigen-specific T-cells and APCs, where it localizes with the TCR in the central core of the T-cell activation. PKCθ, but not the α, ε, or ζ isoenzymes, selectively activated a FasL promoter-reporter gene and upregulated the mRNA or cell surface expression of endogenous FasL. On the other hand, PKCθ and ε promoted T-cell survival by protecting the cells from Fas-induced apoptosis, and this protective effect was mediated by promoting p90Rsk-dependent phosphorylation of BAD. Thus, PKCθ appears to play a dual regulatory role in T-cell apoptosis.

The selective expression of PKCθ in T-cells and its essential role in mature T-cell activation establish that PKCθ inhibitors are useful for the treatment or prevention of disorders or diseases mediated by T lymphocytes, for example autoimmune disease such as rheumatoid arthritis and lupus erythematosus, and inflammatory disease such as asthma, and inflammatory bowel diseases.

PKCθ is identified as a drug target for immunosuppression in transplantation and autoimmune diseases (Isakov et al. (2002) Annual Review of Immunology, 20, 761-794). PCT Publication WO2004/043386 identifies PKCθ as a target for treatment of transplant rejection and multiple sclerosis. PKCθ also plays a role in inflammatory bowel disease (The Journal of Pharmacology and Experimental Therapeutics (2005), 313 (3), 962-982), asthma (WO 2005062918), and lupus (Current Drug Targets: Inflammation & Allergy (2005), 4(3), 295-298).

In addition, PKCθ is highly expressed in gastrointestinal stromal tumors (Blay, P. et al. (2004) Clinical Cancer Research, 10, 12, Pt. 1), it has been suggested that PKCθ is a molecular target for treatment of gastrointestinal cancer (Wiedmann, M. et al. (2005) Current Cancer Drug Targets 5(3), 171). Thus, small molecule PKC-theta inhibitors can be useful for treatment of gastrointestinal cancer.

Experiments conducted in PKCθ knock-out mice led to the conclusion that PKCθ inactivation prevented fat-induced defects in insulin signalling and glucose transport in skeletal muscle (Kim J. et al, 2004, The J. of Clinical Investigation 114 (6), 823). This data suggests that PKCθ is a potential therapeutic target for the treatment of type 2 diabetes, and hence small molecule PKCθ inhibitors can be useful for treating such disease.

Therefore, PKCθ inhibitors are useful in treatment of T-cell mediated diseases including autoimmune disease such as rheumatoid arthritis and lupus erythematosus, and inflammatory diseases such as asthma and inflammatory bowel disease. In addition, PKCθ inhibitors are useful in treatment of gastrointestinal cancer and diabetes.

A variety of structural classes of compounds are known which act as PKCθ inhibitors. For example, Cywin and co-workers recently described 2,4-diamino-5-nitropyrimidines as potent and selective PKCtheta inhibitors (Bio-organic Medicinal Chemistry Letters, 17, 2007, 225-230). WO 2005066139 describes 2-(amino-substituted)-4-aryl pyrimidines useful for treating inflammatory disorders in which PKCtheta plays a role. In addition, WO 2007038519 describes thieno[2,3-B]pyridine-5-carbonitriles that inhibit PKCtheta.

WO 2007047207 relates to indole derivatives indicated to be 5-lipoxygenase activating protein inhibitors and human leukocyte inhibitors. WO 2005044181 relates to azabicyclic compounds indicated to be abelson tyrosine kinase inhibitors. WO 200149688 relates to purine and aza-deaza analogues indicated to be useful as cyclin dependent kinase inhibitors. WO 200443394 relates to substituted nitrogen heterocyclic derivatives having immunological properties. None of these documents teach or suggest compounds having PKCθ inhibitory properties.

In a first aspect the present invention relates to a pyrrolo[2,3-d]pyrimidin-2-yl-amine derivative according to formula I

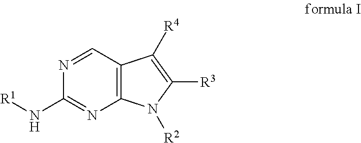

wherein
R¹ is C₆₋₁₀aryl optionally substituted with one or more substituents independently selected from halogen, hydroxy, cyano, C₁₋₆alkyl, C₃₋₆cycloalkyl, C₁₋₆alkyloxy and C₃₋₆cycloalkyloxy, said C₁₋₆alkyl, C₃₋₆ cycloalkyl, C₁₋₆alkyloxy and C₃₋₆cycloalkyloxy being optionally substituted with one or more halogens or
   R¹ is C₃₋₈cycloalkyl or
   R¹ is —C₁₋₃alkyl-Z, wherein Z is C₃₋₈cycloalkyl, C₆₋₁₂aryl or a 5-10 membered heteroaryl ring system comprising 1-2 heteroatoms independently selected from O, S and N, said C₆₋₁₀aryl and 5-10 membered heteroaryl ring system being optionally substituted with one or more substituents independently selected from halogen, hydroxy, cyano, C₁₋₆alkyl, C₃₋₆cycloalkyl, C₁₋₆alkyloxy and C₃₋₆cycloalkyloxy, said C₁₋₆alkyl, C₃₋₆cycloalkyl, C₁₋₆alkyloxy and C₃₋₆cycloalkyloxy being optionally substituted with one or more halogens;
R² is —C₂₋₇alkyl-NR⁵R⁶ or
   R² is —C₀₋₄alkyl-Y wherein Y is a 4-8 membered saturated or unsaturated heterocyclic ring system comprising one or two heteroatomic moieties independently selected from O, S and N(R⁷)ₚ, said heterocyclic ring system being optionally substituted with halogen, hydroxy, C₁₋₆alkyl or C₁₋₆alkyloxy or
   R² is —C₀₋₂alkylC₃₋₆cycloalkyl substituted with —NR⁸R⁹ or —CH₂NR⁸R⁹;
R³ is C₁₋₆alkyl, C₆₋₁₀aryl or C₆₋₁₀arylC₁₋₃alkyl, said C₆₋₁₀aryl and C₆₋₁₀arylC₁₋₃alkyl being optionally substituted with one or more substituents independently selected from halogen, hydroxy, cyano, C₁₋₆alkyl, C₃₋₆cycloalkyl, C₁₋₆alkyloxy, C₃₋₆cycloalkyloxy, —NHCOR¹⁰, —NHS(O)_{q}R¹¹, —CONR¹²R¹³, —S(O)ᵣR¹⁴R¹⁵, and —NHCONR¹⁶R¹⁷ said C₁₋₆alkyl, C₃₋₆ cycloalkyl, C₁₋₆alkyloxy and C₃₋₆cycloalkyloxy being optionally substituted with one or more halogens;
R⁴ is H, C₁₋₆alkyl, CN or halogen;
R⁵-R⁹ are independently chosen from H and C₁₋₄alkyl;
R¹⁰ and R¹¹ are independently C₁₋₄alkyl;
R¹² and R¹³ are independently chosen from H and C₁₋₄alkyl;
R¹⁴-R¹⁷ are independently C₁₋₄alkyl;
p is 0 or 1 and
q and r are independently 1 or 2
or a pharmaceutically acceptable salt or solvate thereof.

The term C₁₋₆alkyl, as used herein, represents a branched or unbranched alkyl group having 1-6 carbon atoms. Examples of such groups are methyl, ethyl, isopropyl, tertiary butyl and isopentyl. Similarly the term C₁₋₄alkyl represents a branched or unbranched alkyl group having 1-4 carbon atoms.

The term C₁₋₃alkyl-Z, as used herein, represents a C₁₋₃alkyl group which is substituted with a Z group, wherein Z has the previously defined meanings. Examples of such groups are cyclohexylmethyl, (4-chlorophenyl)ethyl and (2-chlorothien-3-yl)methyl.

Similarly, the term C₂₋₇alkyl-NR⁵R⁶, as used herein, represents a C₂₋₇alkyl group which is substituted with an amine group of the formula NR⁵R⁶, wherein R⁵ and R⁶ have the previously defined meanings. Examples of such groups are —(CH₂)₃—N(CH₃)₂ and —(CH₂)₅—N(CH₃)₂.

The term —C₀₋₄alkyl-Y, as used herein, represents a C₁₋₄alkyl group which is substituted with a Y group or a Y group itself without an alkyl linking group, wherein Y has the previously defined meanings. Examples of such groups are (pyridine2-yl)methyl and (piperidine-3-yl)methyl.

The term C₃₋₈cycloalkyl, as used herein, represents a branched or unbranched cyclic alkyl group having 3-8 carbon atoms. Examples of such groups are cyclopropyl, cyclopentyl and 2-methylcyclohexyl. Similarly the term C₃₋₆cycloalkyl, as used herein, represents a branched or unbranched cyclic alkyl group having 3-6 carbon atoms. Examples of such groups are cyclopropyl, cyclopentyl and 2-methylcyclopentyl.

The term —C₀₋₂alkyl-C₃₋₆cycloalkyl, as used herein, represents a C₁₋₂alkyl group which is substituted with a C₃₋₆cycloalkyl or a C₃₋₆cycloalkyl group itself without an alkyl linking group. Examples of such groups are cyclopentylmethyl and cyclohexylethyl.

The term C₁₋₆alkyloxy, as used herein, represents a branched or unbranched alkyloxy group having 1-6 carbon atoms. Examples of such groups are methoxy, ethoxy, isopropyloxy and tertiary-butyloxy.

The term C₃₋₆cycloalkyloxy, as used herein, represents a branched or unbranched cyclic alkyloxy group having 3-6 carbon atoms. Examples of such groups are cyclopropyloxy, cyclopentyloxy and 2-methylcyclopentyloxy.

The term C₆₋₁₀aryl, as used herein, represents an aromatic group having 6-10 carbon atoms and comprising one ring or two rings fused together, at least one of which must be aromatic. Examples of such groups include both monocyclic and fused bicyclic aromatic groups e.g., phenyl and naphthyl.

The term C₆₋₁₀arylC₁₋₃alkyl, as used herein, represents a C₁₋₃alkyl group which is substituted with a C₆₋₁₀aryl group. Examples of such groups are benzyl and phenethyl.

5 to 10 Membered heteroaryl ring systems comprising 1-2 heteroatoms independently selected from O, S and N, as used herein, encompass both monocyclic and fused bicyclic systems. Examples of said groups are furan, pyrrole, thiophene, imidazole, pyrrazole, thiazole, pyridine, pyrimidine, indole, indazole and benzthiophene.

Examples of 4 to 8 membered saturated or unsaturated heterocyclic ring systems comprising one or two heteroatomic moieties independently selected from O, S and N(R⁷)ₚ, wherein R⁷ and p have the previously defined meanings are pyrrole, imidazole, pyrrazole, thiazole, pyridine piperidine morpholine and piperazine.

The term solvate, as used herein, refers to a complex of variable stoichiometry formed by a solvent and a solute (in this invention, a compound of formula I). Such solvents may not interfere with the biological activity of the solute. Examples of suitable solvents include, water, ethanol and acetic acid.

In one embodiment of the present invention R¹ is phenyl optionally substituted with one or more substituents independently chosen from halogen, hydroxy, —OCH₃, —CF₃, —OCF₃ and C₁-₄alkyl. In a further embodiment of the present invention R¹ is phenyl optionally substituted with one or more substituents independently selected from chloro, fluoro, methyl, hydroxy and methoxy.

In another embodiment of the present invention R¹ is —C₁₋₃alkyl-Z, wherein Z is phenyl optionally substituted with one or more substituents independently chosen from halogen, hydroxy, —OCH₃, —CF₃, —OCF₃ and C₁-₄alkyl. In a further embodiment of the present invention R¹ is —C₁₋₃alkyl-Z, wherein Z is phenyl optionally substituted with one or more substituents independently selected from chloro, fluoro, methyl, hydroxy and methoxy.

In another embodiment of the present invention R¹ is —CH₂—Z, wherein Z is phenyl optionally substituted with one or more substituents independently chosen from halogen, hydroxy, —OCH₃, —CF₃, —OCF₃ and C₁-₄alkyl. In a further embodiment of the present invention R¹ is —CH₂—Z, wherein Z is phenyl optionally substituted with one or more substituents independently selected from chloro, fluoro, methyl, hydroxy and methoxy.

In another embodiment of the present invention R¹ is C₃₋₈cycloalkyl.

In another embodiment of the present invention R¹ is —C₁₋₃alkyl-Z, wherein Z is a 5-10 membered heteroaryl ring system comprising 1-2 heteroatoms independently selected from O, S and N, said 5-10 membered heteroaryl ring system being optionally substituted with one or two substituents independently chosen from halogen, hydroxy, —OCH₃, —CF₃, —OCF₃ and C₁-₄alkyl. In a further embodiment of the present invention R¹ is —C₁₋₃alkyl-Z, wherein Z is a 5-10 membered heteroaryl ring system comprising 1-2 heteroatoms independently selected from O, S and N, said 5-10 membered heteroaryl ring system being optionally substituted with one or two substituents independently selected from chloro, fluoro, methyl, hydroxy and methoxy.

In another embodiment of the present invention R¹ is —CH₂—Z, wherein Z is a 5-10 membered heteroaryl ring system comprising 1-2 heteroatoms independently selected from O, S and N, said 5-10 membered heteroaryl ring system being optionally substituted with one or two substituents independently chosen from halogen, hydroxy, —OCH₃, —CF₃, —OCF₃ and C₁-₄alkyl. In a further embodiment of the present invention R¹ is —CH₂—Z, wherein Z is a 5-10 membered heteroaryl ring system comprising 1-2 heteroatoms independently selected from O, S and N, said 5-10 membered heteroaryl ring system being optionally substituted with one or two substituents independently selected from chloro, fluoro, methyl, hydroxy and methoxy.

In another embodiment of the present invention R¹ is —CH₂—Z, wherein Z is thienyl said thienyl being optionally substituted with one or two substituents independently chosen from halogen, hydroxy, —OCH₃, —CF₃, —OCF₃ and C₁-₄alkyl. In a further embodiment of the present invention R¹ is —CH₂—Z, wherein Z is thienyl, said thienyl being optionally substituted with one or two substituents independently selected from chloro, fluoro, methyl, hydroxy and methoxy.

In another embodiment of the present invention R² is —C₂₋₇alkyl-NR⁵R⁶. In a further embodiment of the present invention R² is —(CH₂)₂—NR⁵R⁶.

In another embodiment of the present invention, R² is —C₀₋₄alkyl-Y, wherein Y is a 4-8 membered saturated or unsaturated heterocyclic ring comprising one or two heteroatomic moieties independently selected from O, S and N(R⁷)ₚ, said heterocyclic ring being optionally substituted with halogen, hydroxy, C₁₋₆alkyl or C₁₋₆alkyloxy.

In another embodiment of the present invention, R² is —CH₂—Y, wherein Y is a 4-8 membered saturated or unsaturated heterocyclic ring comprising one or two heteroatomic moieties independently selected from O, S and N(R⁷)ₚ, said heterocyclic ring being optionally substituted with halogen, hydroxy, C₁₋₆alkyl or C₁₋₆alkyloxy. In another embodiment, R² is —CH₂Y, wherein Y is piperidinyl, morpholinyl or pyrrolidinyl.

In another embodiment of the present invention R² is —C₀₋₂alkylC₃₋₆cycloalkyl substituted with —NR⁸R⁹ or —C₁₋₂alkylNR⁸R⁹, wherein R⁸ and R⁹ have the previously defined meanings. In a further embodiment of the present invention R² is —CH₂C₃₋₆cycloalkyl substituted with —NR⁸R⁹, wherein R⁸ and R⁹ have the previously defined meanings.

In another embodiment of the present invention R² is a group selected from:

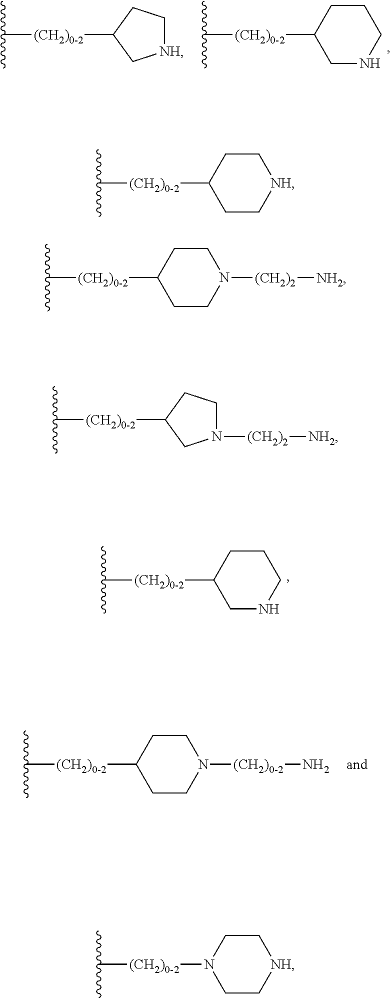

In a further embodiment of the present invention R² is a group selected from:

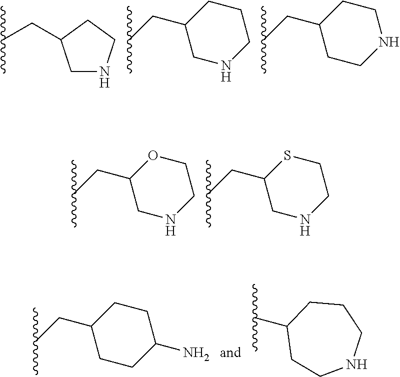

In another embodiment of the present invention R³ is phenyl optionally substituted with one or more substituents independently chosen from halogen, hydroxy, —OCH₃, —OF₃, —OCF₃ CN and C₁-₄alkyl. In a further embodiment of the present invention R³ is phenyl optionally substituted with one or more substituents independently selected from chloro, fluoro, methyl, hydroxy and methoxy.

In a further embodiment of the present invention R³ is —CH₂-phenyl optionally substituted with one or more substituents independently chosen from halogen, hydroxy, —OCH₃, —OF₃, —OCF₃ CN and C₁-₄alkyl. In a further embodiment of the present invention R³ is —CH₂-phenyl optionally substituted with one or more substituents independently selected from chloro, fluoro, methyl, hydroxy and methoxy.

In another embodiment of the present invention R⁴ is H or methyl.

In another embodiment R⁴ is halogen. In a further embodiment, R⁴ is fluoro or chloro.

In another embodiment, R⁴ is nitrile.

In another embodiment of the present invention R⁵ is H or methyl.

In another embodiment of the present invention R⁶ is H or methyl.

In another embodiment of the present invention R⁷ is H or methyl.

In another embodiment of the present invention R⁸ is H or methyl.

In another embodiment of the present invention R⁹ is H or methyl.

In another embodiment of the present invention p is 0. In another embodiment of the present invention p is 1.

In another embodiment of the present invention q is 1. In another embodiment of the present invention q is 2.

In another embodiment of the present invention r is 1. In another embodiment of the present invention r is 2.

In another embodiment of the present invention is a pyrrolo[2,3-d]pyrimidin-2-yl-amine derivative having the general formula VI,

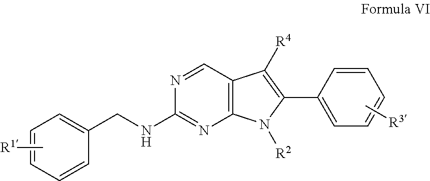

wherein

R^{1′} is one or more chloro, bromo, fluoro, methyl, hydroxy or methoxy;

R² is

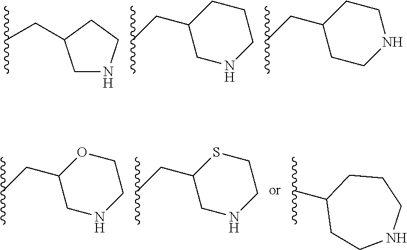

R^{3′} is chloro, fluoro, methyl, hydroxy or methoxy and

R⁴ is H, methyl, CN or halogen

or a pharmaceutically acceptable salt or solvate thereof.

In another embodiment of the present invention is a pyrrolo[2,3-d]pyrimidin-2-yl-amine derivative selected from:

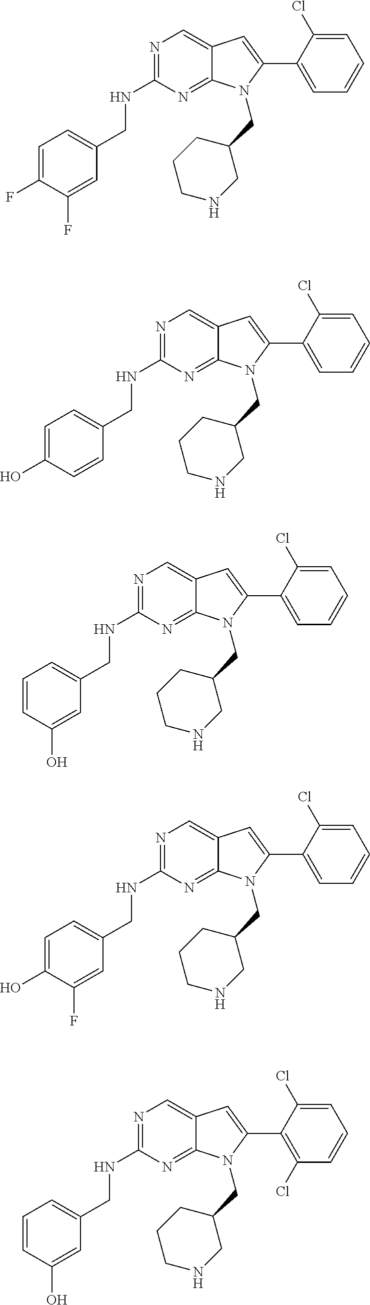

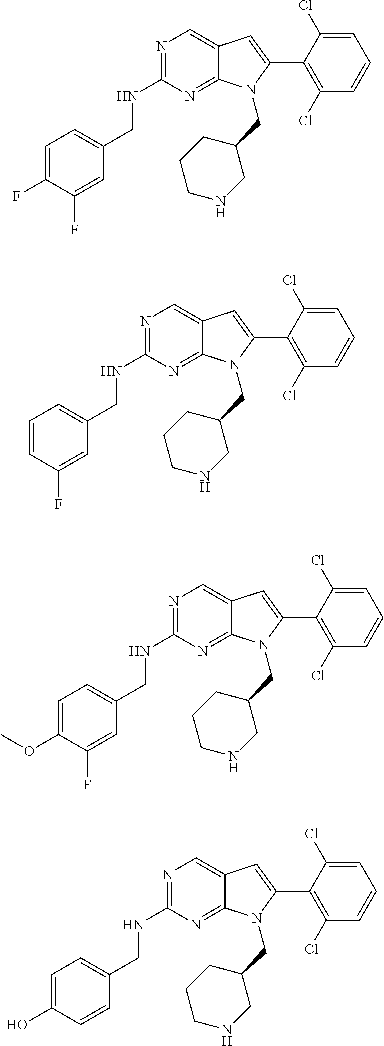

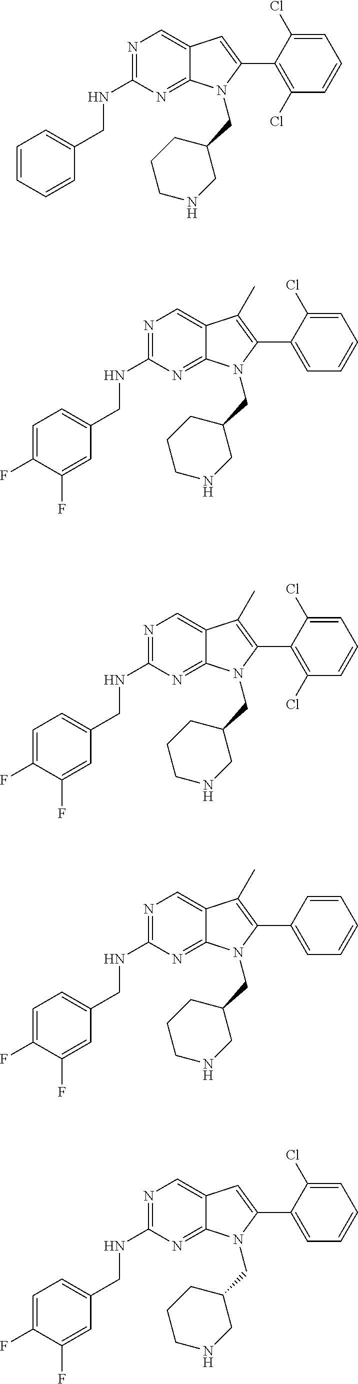

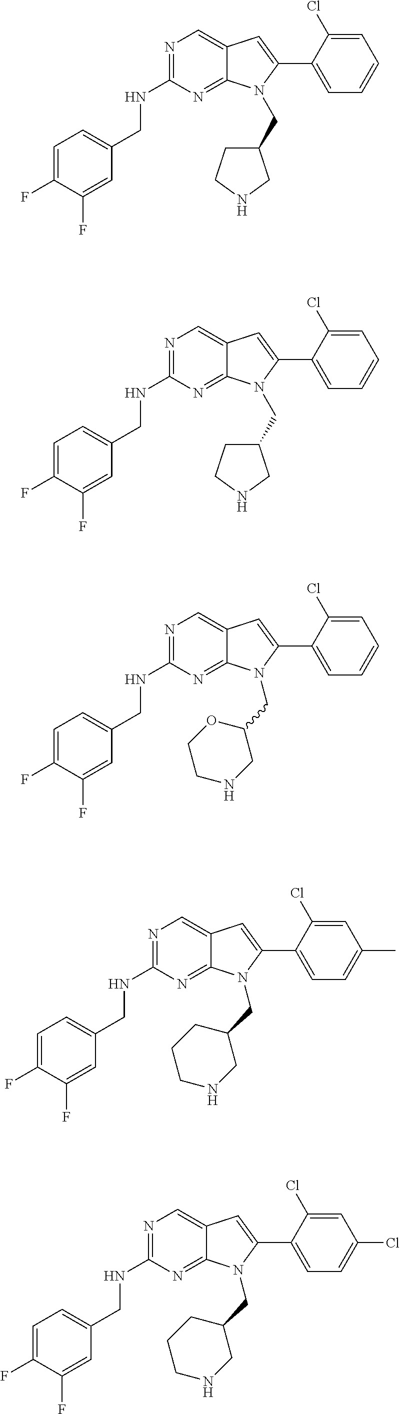

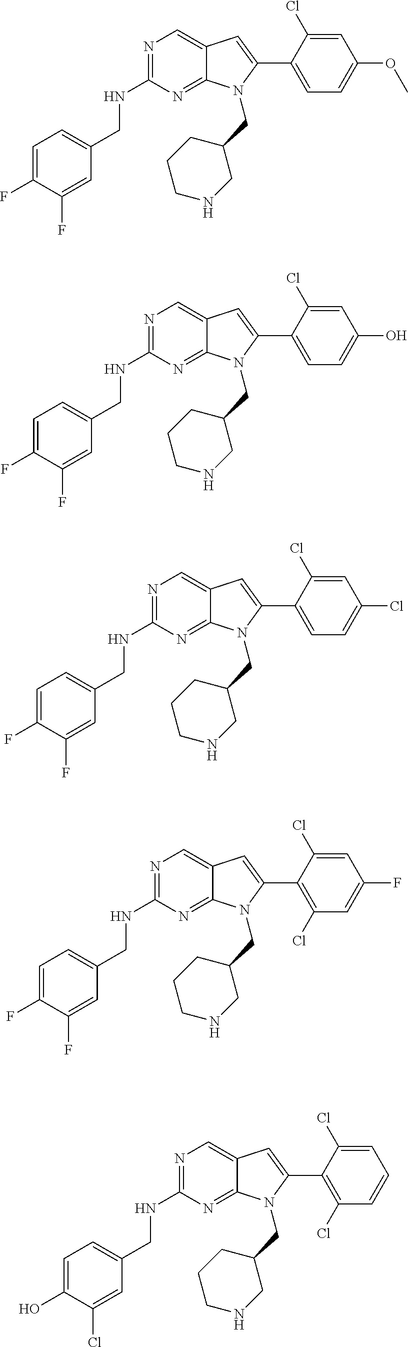

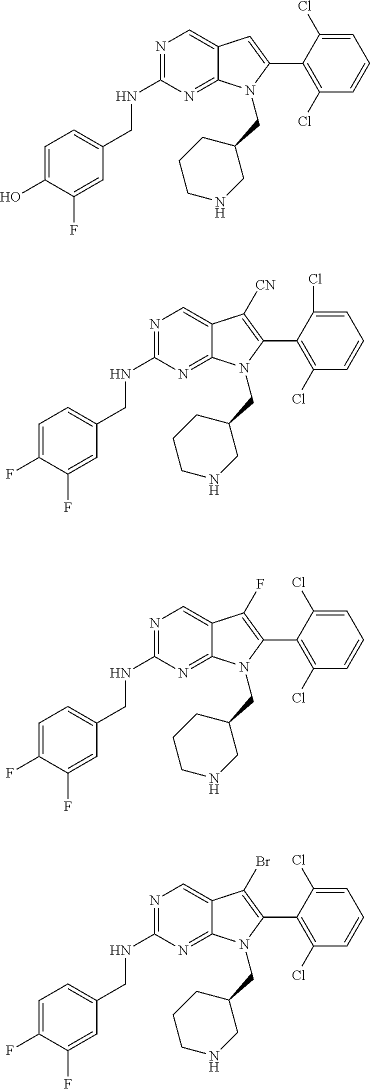

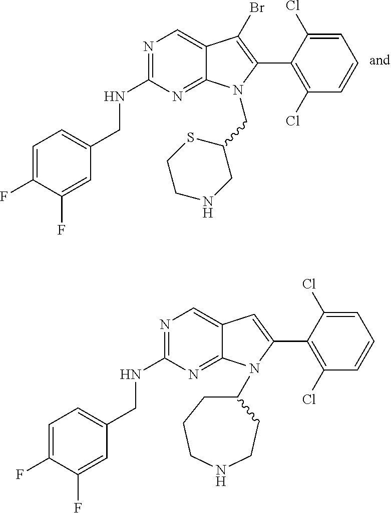

or a pharmaceutically acceptable salt or solvate thereof

The pyrrolo[2,3-d]pyrimidine-2-yl-amine derivatives of the present invention can be prepared by methods well known in the art of organic chemistry. See, for example, J. March, ‘Advanced Organic Chemistry’ 4^{th} Edition, John Wiley and Sons. During synthetic sequences it may be necessary and/or desirable to protect sensitive or reactive groups on any of the molecules concerned. This is achieved by means of conventional protecting groups, such as those described in T. W. Greene and P. G. M. Wutts ‘Protective Groups in Organic Synthesis’ 2^{nd} Edition, John Wiley and Sons, 1991. The protective groups are optionally removed at a convenient subsequent stage using methods well known in the art.

Pyrrolo[2,3-d]pyrimidine-2-yl-amine derivatives of formula I, wherein R¹-R⁴ have the previously defined meanings, can be prepared by the general synthetic route shown in scheme I.

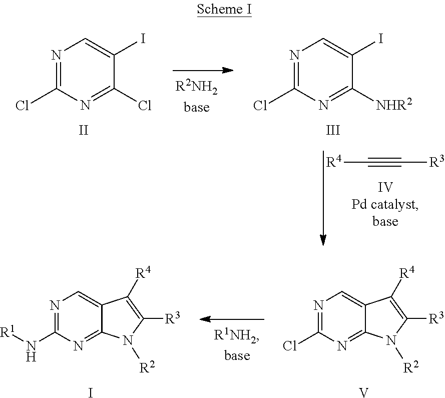

Treatment of 2,4-dichloro-5-iodopyrimidine (II) with an appropriately functionalised amine R²NH₂ in the presence of a suitable base and solvent, for example diisopropylethylamine in teterahydrofuran provides the adduct III. This can then be reacted with an appropriately substituted acetylene (IV), in the presence of a suitable palladium catalyst system and solvent, for example tetrakis(triphenylphosphine)palladium (0) and copper iodide in the presence of diisopropylethylamine in N,N-dimethylformamide, followed by treatment with potassium tertiary-butoxide to effect cyclisation to the desired pyrrolopyrimidine V. Finally treatment of pyrrolopyrimidine V with an appropriately functionalised amine R¹NH₂ in the presence of a suitable base and solvent, for example diisopropylethylamine in teterahydrofuran provides the pyrrolopyrimidin-2-amine I.

The amines R¹NH₂ and R²NH₂ are either commercially available or they can be readily prepared using methods well known to the skilled organic chemist. For example the amines R¹NH₂, wherein R¹ is ZCH₂ and wherein Z has the previously defined meanings are commercially available or can be readily prepared by reaction of the appropriate alkyl halide ZCH₂Cl or ZCH₂Br with a protected amine, followed by removal of the protecting group. For example, compounds of the form ZCH₂NH₂ can readily be prepared by reaction of precursors of the formula ZCH₂Br with sodium azide followed by reduction with a suitable reducing agent, for example with lithium aluminium hydride. Similarly the amines R²NH₂, wherein R² is (CH₂)₃NR⁵R⁶, wherein R⁵ and R⁶ have the previously defined meanings are commercially available or they can readily be prepared by, for example, reaction of 3-bromopropylphthalimide with the amine NHR⁵R⁶ followed by removal of the phthalimide protecting group with, for example, hydrazine hydrate in ethanol.

The substituted acetylenes (IV) are also readily prepared by methods well known to the skilled organic chemist. For example, the acetylene, wherein R⁴ is H and R³ is aryl (Ar) may be prepared by reaction of trimethylsilylacetylene with ArX, wherein X is a suitable leaving group such as a triflate, with a suitable palladium catalyst, for example, bis(triphenylphosphine)palladium(II) chloride in the presence of a suitable base and solvent, for example, triethylamine in N,N-dimethylformamide. Acetylenes having R⁴ as C₁₋₄alkyl can be readily prepared from the acetylenes wherein R⁴ is H by standard alkylation reactions well known to the skilled organic chemist, for example, by treatment of the acetylene, wherein R³ is aryl and R⁴ is H with a base, for example n-butyl lithium followed by reaction with an alkyl halide, for example methyl iodide in a suitable solvent, for example, tetrahydrofuran.

Palladium catalysts and conditions to form either the acetylene or to couple the acetylene with the iodopyrimidine are well known to the skilled organic chemist—see, for example, Ei-ichi Negishi (Editor), Armin de Meijere (Associate Editor), Handbook of Organopalladium Chemistry for Organic Synthesis, John Wiley and Sons, 2002.

Pyrrolo[2,3-d]pyrimidine-2-yl-amine derivatives of formula I, wherein R¹ is (substituted) benzyl may be further manipulated by removal of the benzyl group, for example, by treatment with dichlorodicyanoquinone in methylene chloride and thereafter further functionalisation of the resulting free amino group. For example the free amino group may be functionalised by reductive alkylation, for example, by reacting with an appropriate aldehyde in the presence of sodium triacetoxy borohydride in a suitable solvent, for example, ethanol.

The present invention also includes within its scope all stereoisomeric forms of the pyrrolo[2,3-d]pyrimidine-2-yl-amine derivatives according to the present invention resulting, for example, because of configurational or geometrical isomerism. Such stereoisomeric forms are enantiomers, diastereoisomers, cis and trans isomers etc. For example where R² is (piperidin-3-yl)methyl, there exists a mixture of two enantiomers. In the case of the individual stereoisomers of heterocyclic derivatives of formula I or salts or solvates thereof, the present invention includes the aforementioned stereoisomers substantially free, i.e., associated with less than 5%, preferably less than 2% and in particular less than 1% of the other stereoisomer. Mixtures of stereoisomers in any proportion, for example a racemic mixture comprising substantially equal amounts of two enantiomers are also included within the scope of the present invention.

For chiral compounds, methods for asymmetric synthesis whereby the pure stereoisomers are obtained are well known in the art, e.g., synthesis with chiral induction, synthesis starting from chiral intermediates, enantioselective enzymatic conversions, separation of stereoisomers using chromatography on chiral media. Such methods are described in Chirality In Industry (edited by A. N. Collins, G. N. Sheldrake and J. Crosby, 1992; John Wiley). Likewise methods for synthesis of geometrical isomers are also well known in the art.

The pyrrolo[2,3-d]pyrimidine-2-yl-amine derivatives of the present invention, in the form as a free base, are isolated from reaction mixtures as pharmaceutically acceptable salts. These salts are also obtained by treatment of said free base with an organic or inorganic acid, for example, hydrogen chloride, hydrogen bromide, hydrogen iodide, sulfuric acid, phosphoric acid, acetic acid, trifluoroacetic acid, propionic acid, glycolic acid, maleic acid, malonic acid, methanesulfonic acid, fumaric acid, succinic acid, tartaric acid, ciric acid, benzoic acid and ascorbic acid.

The pyrrolo[2,3-d]pyrimidine-2-yl-amine derivatives of the present invention also exist as amorphous forms. Multiple crystalline forms are also possible. All these physical forms are included within the scope of the present invention.

Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS Pharm Sci Tech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labelled compounds of Formula I (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labelled compounds of Formula (I can generally be prepared by following procedures analogous to those disclosed in the Schemes and/or in the Examples hereinbelow, by substituting an appropriate isotopically labelled reagent for a non-isotopically labelled reagent.

In a further aspect, the pyrrolo[2,3-d]pyrimidine-2-yl-amine derivatives of the present invention and their pharmaceutically acceptable salts and solvates are useful in therapy. As such the pyrrolo[2,3-d]pyrimidine-2-yl-amine derivatives of the present invention are useful for the treatment of PKCθ mediated disorders. In particular the pyrrolo[2,3-d]pyrimidine-2-yl-amine derivatives are useful for treatment of arthritis (such as rheumatoid arthritis, psoriatic arthritis or osteoarthritis); transplant rejection (such as organ transplant, acute transplant or heterograft or homograft (such as is employed in burn treatment)); protection from ischemic or reperfusion injury such as ischemic or reperfusion injury incurred during organ transplantation, myocardial infarction, stroke or other causes; transplantation tolerance induction; multiple sclerosis; inflammatory bowel disease, including ulcerative colitis and Crohn's disease; lupus (systemic lupus erythematosis); graft vs. host diseases; T cell mediated hypersensitivity diseases, including contact hypersensitivity, delayed-type hypersensitivity and gluten-sensitive enteropathy (Celiac disease); Type 1 diabetes; psoriasis; contact dermatitis (including that due to poison ivy); Hashimomoto's thyroiditis; Sjogren's syndrome; Autoimmune Hyperthyroidism, such as Graves' Disease; Addison's Disease (autoimmune disease of the adrenal glands); Autoimmune polyglandular disease (also known as autoimmune polyglandular syndrome); autoimmune alopecia; pernicious anaemia; vitiligo; autoimmune hypopituatarism; Guillain-Barre syndrome; other autoimmune diseases; cancers where PKCtheta are activated or overexpressed, or cancers where PKCtheta kinase activity facilitates tumor growth or survival or provides resistance to chemotherapeutic drugs or radiation; glomerulonephritis, serum sickness; uticaria; allergic diseases such as respiratory allergies (asthma, hay fever, allergic rhinitis) or skin allergies; scleracielma; mycosis fungoides; acute inflammatory responses (such as acute respiratory distress syndrome and ischemia/reperfusion injury); dermatomyositis; alopecia areata; chronic actinic dermatitis; eczema; Behcet's disease; Pustulosis palmoplanteris; Pyoderma gangrenum; Sezary's syndrome; atopic dermatitis; systemic sclerosis; morphea; Type II diabetes; insulin resistance; diabetic retinopathy; diabetic macular edema; diabetic neuropathy; cardiovascular disease in diabetic patients.

The present invention further includes a method for the treatment of a mammal, including a human, suffering from or liable to suffer from depression or any of the aforementioned disorders, which comprises administering an effective amount of a pyrrolo[2,3-d]pyrimidine-2-yl-amine derivative according to the present invention or a pharmaceutically acceptable salt or solvate thereof. By effective amount or therapeutically effective amount is meant an amount of compound or a composition of the present invention effective in inhibiting the above-noted diseases and thus producing the desired therapeutic, ameliorative, inhibitory or preventative effect.

The amount of a pyrrolo[2,3-d]pyrimidine-2-yl-amine derivative of the present invention or a pharmaceutically acceptable salt or solvate thereof, also referred to herein as the active ingredient, which is required to achieve a therapeutic effect will, of course, vary with the particular compound, the route of administration, the age and condition of the recipient and the particular disorder or disease being treated.

A suitable daily dose for any of the above mentioned disorders will be in the range of 0.001 to 50 mg per kilogram body weight of the recipient (e.g. a human) per day, preferably in the range of 0.01 to 20 mg per kilogram body weight per day. The desired dose may be presented as multiple sub-doses administered at appropriate intervals throughout the day.

Whilst it is possible for the active ingredient to be administered alone, it is preferable to present it as a pharmaceutical composition. The present invention therefore also provides a pharmaceutical composition comprising a pyrrolo[2,3-d]pyrimidine-2-yl-amine derivative according to the present invention in admixture with one or more pharmaceutically acceptable excipients, such as the ones described in Gennaro et. al., Remmington: The Science and Practice of Pharmacy, 20^{th} Edition, Lippincott, Williams and Wilkins, 2000; see especially part 5: pharmaceutical manufacturing. The term “acceptable” means being compatible with the other ingredients of the composition and not deleterious to the recipients thereof. Suitable excipients are described e.g., in the Handbook of Pharmaceutical Excipients, 2^{nd} Edition; Editors A. Wade and P. J. Weller, American Pharmaceutical Association, Washington, The Pharmaceutical Press, London, 1994. Compositions include those suitable for oral, nasal, topical (including buccal, sublingual and transdermal), parenteral (including subcutaneous, intravenous and intramuscular) or rectal administration.

The mixtures of a pyrrolo[2,3-d]pyrimidine-2-yl-amine derivative according to the present invention and one or more pharmaceutically acceptable excipient or excipients may be compressed into solid dosage units, such as tablets, or be processed into capsules or suppositories. By means of pharmaceutically suitable liquids the compounds can also be applied as an injection preparation in the form of a solution, suspension, emulsion, or as a spray, e.g., a nasal or buccal spray. For making dosage units e.g., tablets, the use of conventional additives such as fillers, colorants, polymeric binders and the like is contemplated. In general, any pharmaceutically acceptable additive can be used. The compounds of the invention are also suitable for use in an implant, a patch, a gel or any other preparation for immediate and/or sustained release.

Suitable fillers with which the pharmaceutical compositions can be prepared and administered include lactose, starch, cellulose and derivatives thereof, and the like, or mixtures thereof used in suitable amounts. For parenteral administration, aqueous suspensions, isotonic saline solutions and sterile injectable solutions may be used, containing pharmaceutically acceptable dispersing agents and/or wetting agents, such as propylene glycol or butylene glycol.

The present invention further includes a pharmaceutical composition, as hereinbefore described, in combination with packaging material suitable for said composition, said packaging material including instructions for the use of the composition for the use as hereinbefore described.

The invention is further illustrated by the following examples which are not intended to limit the scope thereof. Unless otherwise indicated, percent is percent by weight given the component and the total weight of the composition, temperature is in ° C. or is at ambient temperature and pressure is at or near atmospheric. Commercial reagents were used without further purification. All structures were named using the ‘Convert Structure to Name’-function in Cambridgesoft ChemDraw Ultra version 9.0.7.

Abbreviations

Acetonitrile (ACN), dichloromethane (DCM), 1,2-dicarbonitrile-4,5-dichloro-3,6,dioxo-1,4-cyclohexadiene (DDQ), N,N-diisopropylethyl amine (DIEA), diisopropylazodicarboxylate (DIAD), N,N-dimethylformamide (DMF), dimethylsulfoxide (DMSO), ethyl acetate (EtOAc), hour (h), high performance liquid chromatography (HPLC), liquid chromatography-mass spectrometry (LC-MS), methyl (Me), minutes (min.), mass spectrometry—electrospray ionization MS(ESI), N-methyl-2-pyrrolidinone (NMP), overnight (o.n.), reaction mixture (r.m.), room temperature (r.t.), saturated (satd.), silica (SiO₂), solution (sol), tetrahydrofuran (THF), triethylamine (TEA), triflate (Tf), trifluoroacetic acid (TFA), tert-butyloxycarbonyl (Boc), ultra performance liquid chromatography with mass spectrometry (UPLC-MS) and ultraviolet (UV).

MS(ESI) spectra were obtained using an Applied Biosystems API-165 single quad MS in alternating positive and negative ion mode using Flow Injection. The mass range was 120-2000 Da and scanned with a step rate of 0.2 Da and the capillary voltage was set to 5000 V. Nitrogen gas was used for nebulisation.

LC-MS spectra were obtained using a Waters LC-MS spectrometer with a Chromolith Performance, RP-18e, 4.6×100 mm, XBridge C18, 3.5 μm, 4.6×20 mm column. A standard runtime of 6 minutes was used, with a gradient of 100% (CH₃CN/water—1/9 with 0.05% TFA) to 100% (CH₃CN/water—9/1 with 0.05% TFA) in 3.60 minutes, then 0.05 minutes isocratic at 100% (CH₃CN/water—9/1 with 0.05% TFA), subsequently in 0.35 minutes to 100% (CH₃CN/water—1/9 with 0.05% TFA) and finally 2.00 minutes isocratic at 100% (CH₃CN/water—1/9 with 0.05% TFA). A detector of type PDA (200-320 nm) was used for UV-detection and mass detection was performed with a ZQ-detector.

UPLC-MS data were obtained using a Water acquity UPLC system with a BEH C18 1.7 μm, 2.1×100 mm, XBridge C18, 3.5 μm, 4.6×20 mm column. A standard runtime of 3.70 minutes was used, with a gradient of 100% water (with 0.035% TFA) to 60% CH₃CN in water (with 0.035% TFA) in 3.00 minutes, then in 0.20 minutes to 100% CH₃CN (with 0.035% TFA) and keeping it isocratic at 100% CH₃CN (with 0.035% TFA) for 0.49 minutes and finally, in 0.01 minutes to 100% water (with 0.035% TFA). A detector of type PDA (200-320 nm) was used for UV-detection and mass detection was performed with an SQD-detector.

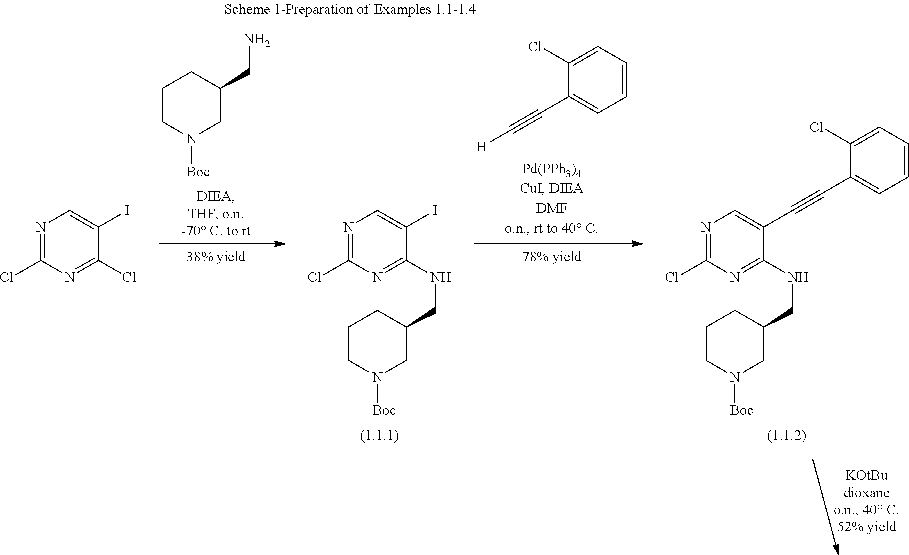

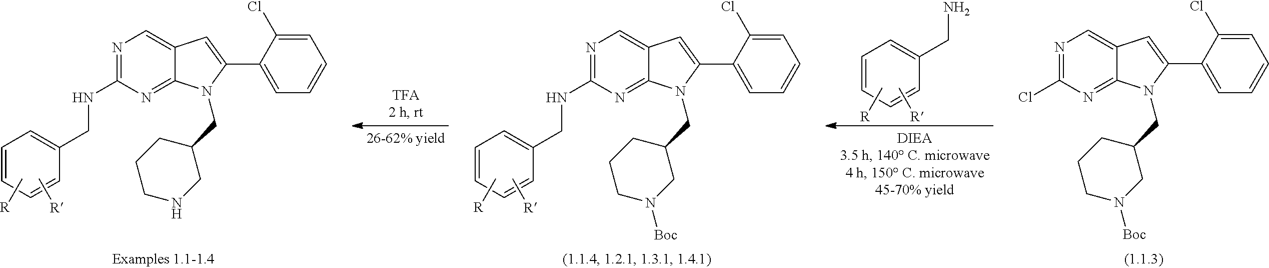

### EXAMPLE 1.1

### (R)-6-(2-chlorophenyl)-N-(3,4-difluorobenzyl)-7-(piperidin-3-ylmethyl)-7H-pyrrolo[2,3-d]pyrimidin-2-amine

### (1.1.1) (S)-tert-butyl 3-((2-chloro-5-iodopyrimidin-4-ylamino)methyl)piperidine-1-carboxylate

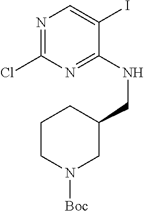

To a stirred solution of 2,4-dichloro-5-iodopyrimidine (3.5 g, 12.73 mmol) in THF (50 mL) at −70° C. was added dropwise a solution of (3S)-aminomethyl-1-piperidinecarboxylic acid-(1,1-dimethyl)ethyl ester (3.0 g, 14 mmol) and DIEA (2.88 mL, 16.55 mmol) in THF (50 mL). The reaction mixture was allowed to warm up to room temperature overnight. Then, the reaction mixture was diluted with EtOAc and washed with saturated NH₄Cl solution (2×) and brine (1×). The organic layer was dried (Na₂SO₄), filtered and concentrated in vacuo. The crude product was purified by column chromatography (SiO₂, heptane/EtOAc; 100% heptane to 30% EtOAc as mobile phase) and the title compound was obtained as a white solid in 38% yield (2.19 g, 4.84 mmol). LC-MS: peak at 4.04 min., mass [M+H]=453.

### (1.1.2) (S)-tert-butyl 3-((2-chloro-5-((2-chlorophenyl)ethynyl)pyrimidin-4-ylamino)methyl)piperidine-1-carboxylate

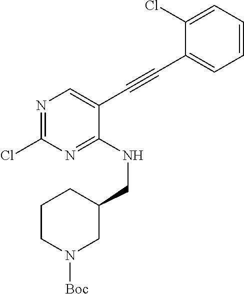

A nitrogen flow was led through a stirred solution of compound 1.1.1 (300 mg, 0.663 mmol) in DMF (6 mL) at room temperature. Subsequently 1-chloro-2-ethynylbenzene (136 mg, 0.99 mmol), DIEA (0.23 mL, 1.33 mmol), copper(I) iodide (3.8 mg, 0.02 mmol) and tetrakis(triphenylphosphine)palladium(0) were added and the reaction mixture was stirred at room temperature over the weekend. Then, the reaction mixture was diluted with EtOAc and washed with water (2×) and brine (1×). The organic layer was dried (Na₂SO₄), filtered and concentrated in vacuo. The crude product was purified by column chromatography (SiO₂, toluene/EtOAc; 100% toluene to 8% EtOAc as mobile phase) and the title compound was obtained as a slightly yellow solid in 78% yield (240 mg, 0.52 mmol). LC-MS: peak at 4.82 min., mass [M+H]=461.

### (1.1.3) (S)-tert-butyl 3-((2-chloro-6-(2-chlorophenyl)-7H-pyrrolo[2,3-d]pyrimidin-7-yl)methyl)piperidine-1-carboxylate

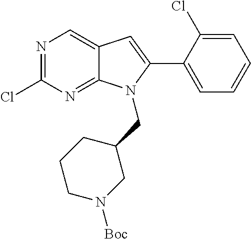

To a stirred solution of compound 1.1.2 (197 mg, 0.43 mmol) in dioxane (15 mL) at room temperature was added potassium tert-butoxide (96 mg, 0.85 mmol). Subsequently the reaction mixture was heated to 40° C. and stirred overnight. Then, the reaction mixture was diluted with DCM and washed with saturated NH₄Cl solution (1×), water (1×) and brine (1×). The organic layer was dried (Na₂SO₄), filtered and concentrated in vacuo. The crude product was purified by column chromatography (SiO₂, heptane/EtOAc; 100% heptane to 30% EtOAc as mobile phase) and the title compound was obtained as a slightly yellow solid in 52% yield (103 mg, 0.22 mmol). LC-MS: peak at 4.48 min., mass [M+H]=461.

### (1.1.4) (S)-tert-butyl 3-((6-(2-chlorophenyl)-2-(3,4-difluorobenzylamino)-7H-pyrrolo[2,3-d]pyrimidin-7-yl)methyl)piperidine-1-carboxylate

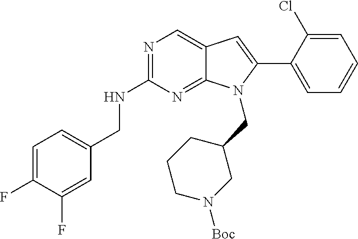

Compound 1.1.3 (100 mg, 0.22 mmol) was dissolved in a mixture of DIEA (1 mL) and 3,4-difluorobenzylamine (1 mL). Subsequently the reaction mixture was heated in the microwave for 3.5 h at 140° C. and another 4 h at 150° C. Then, the reaction mixture was diluted with DCM and washed with saturated NH₄Cl solution (3×) and brine (1×). The organic layer was dried (Na₂SO₄), filtered and concentrated in vacuo. The crude product was purified by column chromatography (SiO₂, heptane/EtOAc; 100% heptane to 25% EtOAc as mobile phase) and the title compound was obtained as colorless oil in 70% yield (87 mg, 0.15 mmol). LC-MS: peak at 3.99 min., mass [M+H]=568.

### (1.1) (R)-6-(2-chlorophenyl)-N-(3,4-difluorobenzyl)-7-(piperidin-3-ylmethyl)-7H-pyrrolo[2,3-d]pyrimidin-2-amine

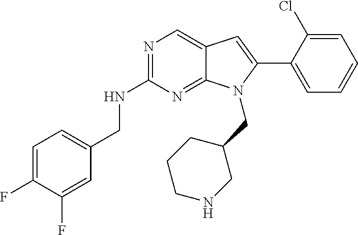

To a stirred solution of compound 1.1.4 (87 mg, 0.15 mmol) in DCM (2 mL) was added trifluoroacetic acid (0.60 mL, 7.66 mmol) at room temperature. The reaction mixture was stirred for 2 h and was then concentrated in vacuo. The crude product was purified by preparative HPLC (0-50% ACN with TFA, as mobile phase). Product fractions were combined, concentrated in vacuo and lyophilized from water/ACN and the TFA-salt of compound the title compound was obtained in 62% yield (55 mg). LC-MS: peak at 2.95 min., mass [M+H]=468.

### EXAMPLE 1.2

### (R)-4-((6-(2-chlorophenyl)-7-(piperidin-3-ylmethyl)-7H-pyrrolo[2,3-d]pyrimidin-2-ylamino)methyl)phenol

### (1.2.1) (S)-tert-butyl 3-((6-(2-chlorophenyl)-2-(4-hydroxybenzylamino)-7H-pyrrolo[2,3-d]pyrimidin-7-yl)methyl)piperidine-1-carboxylate

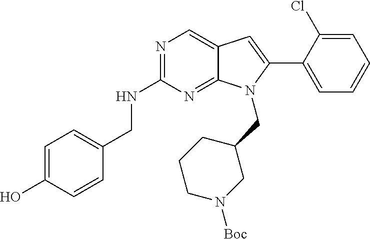

To a solution of compound 1.1.3 (100 mg, 0.22 mmol) in NMP (0.5 mL) were added 4-hydroxybenzylamine (534 mg, 4.3 mmol) and DIEA (76 uL, 0.44 mmol). The reaction mixture was heated in the microwave for 4 hours at 150° C. The reaction mixture was then diluted with DCM and washed with NH₄Cl solution (2×), water (2×) and brine. The organic layer was dried (Na₂SO₄), filtered and concentrated in vacuo. The crude product was purified by column chromatography (SiO₂, heptane/EtOAc; 50% EtOAc as mobile phase) and the title compound was obtained as a solid in 66% yield (79 mg, 0.14 mmol). LC-MS: peak at 3.67 min., mass [M+H]=548.

### (1.2) (R)-4-((6-(2-chlorophenyl)-7-(piperidin-3-ylmethyl)-7H-pyrrolo[2,3-d]pyrimidin-2-ylamino)methyl)phenol

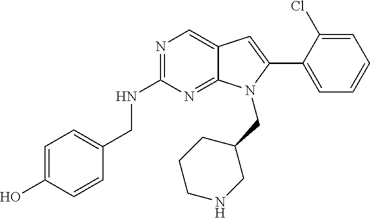

To a stirred solution of compound 1.2.1 (79 mg, 0.14 mmol) in DCM (1 mL) was added TFA (0.5 mL). The reaction mixture was stirred at room temperature for 30 minutes and was subsequently concentrated in vacuo. The crude product was purified by preparative HPLC (0-50% ACN with TFA, as mobile phase). Product fractions were concentrated and lyophilized to obtain the TFA-salt of the title compound in 33% yield (31 mg, 0.05 mmol). LC-MS: peak at 2.72 min., mass [M+H]=448.

### EXAMPLE 1.3

### (R)-3-((6-(2-chlorophenyl)-7-(piperidin-3-ylmethyl)-7H-pyrrolo[2,3-d]pyrimidin-2-ylamino)methyl)phenol

### (1.3.1) (S)-tert-butyl 3-((6-(2-chlorophenyl)-2-(3-hydroxybenzylamino)-7H-pyrrolo[2,3-d]pyrimidin-7-yl)methyl)piperidine-1-carboxylate

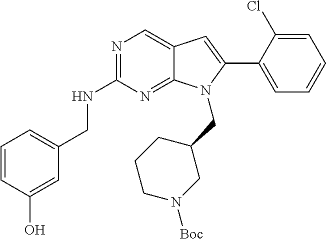

An analogous procedure to that described above to prepare compound 1.2.1 was used except that 3-hydroxybenzylamine was used instead of 4-hydroxybenzylamine. Yield=45% (50 mg, 0.09 mmol). LC-MS: peak at 3.64 min., mass [M+H]=548.

### (1.3) (R)-3-((6-(2-chlorophenyl)-7-(piperidin-3-ylmethyl)-7H-pyrrolo[2,3-d]pyrimidin-2-ylamino)methyl)phenol

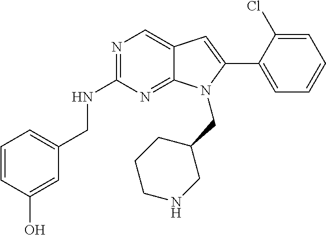

An analogous procedure to that described above to prepare compound 1.2 was used except that compound 1.3.1 was used instead of compound 1.2.1. Yield=60% (31 mg, 0.06 mmol). LC-MS: peak at 2.69 min., mass [M+H]=448.

### EXAMPLE 1.4

### (R)-4-((6-(2-chlorophenyl)-7-(piperidin-3-ylmethyl)-7H-pyrrolo[2,3-d]pyrimidin-2-ylamino)methyl)-2-fluorophenol

### (1.4.1) (S)-tert-butyl 3-((6-(2-chlorophenyl)-2-(3-fluoro-4-methoxybenzylamino)-7H-pyrrolo[2,3-d]pyrimidin-7-yl)methyl)piperidine-1-carboxylate

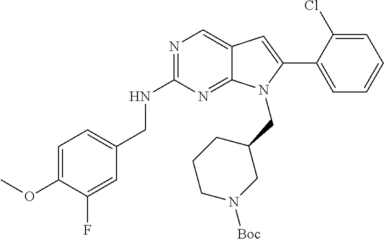

An analogous procedure to that described above to prepare compound 1.2.1 was used except that 3-fluoro-4-methoxybenzylamine was used instead of 4-hydroxybenzylamine. Yield=56% (66 mg, 0.11 mmol). LC-MS: peak at 3.88 min., mass [M+H]=580.

### (1.4) (R)-4-((6-(2-chlorophenyl)-7-(piperidin-3-ylmethyl)-7H-pyrrolo[2,3-d]pyrimidin-2-yl-amino)methyl)-2-fluorophenol

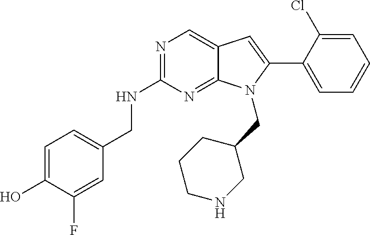

To a solution of compound 1.4.1 (66 mg, 0.11 mmol) in dichloromethane (5 mL) was added boron trifluoride-methyl sulfide complex (36 μl, 0.34 mmol). The reaction mixture was stirred at room temperature for 4 hours. Then, the reaction mixture was concentrated in vacuo and crude product was purified by preparative HPLC (0-50% ACN with TFA, as mobile phase). Product fractions were concentrated and lyophilized to obtain the TFA-salt of the title compound in 26% yield (17 mg, 0.03 mmol). LC-MS: peak at 2.71 min., mass [M+H]=466.

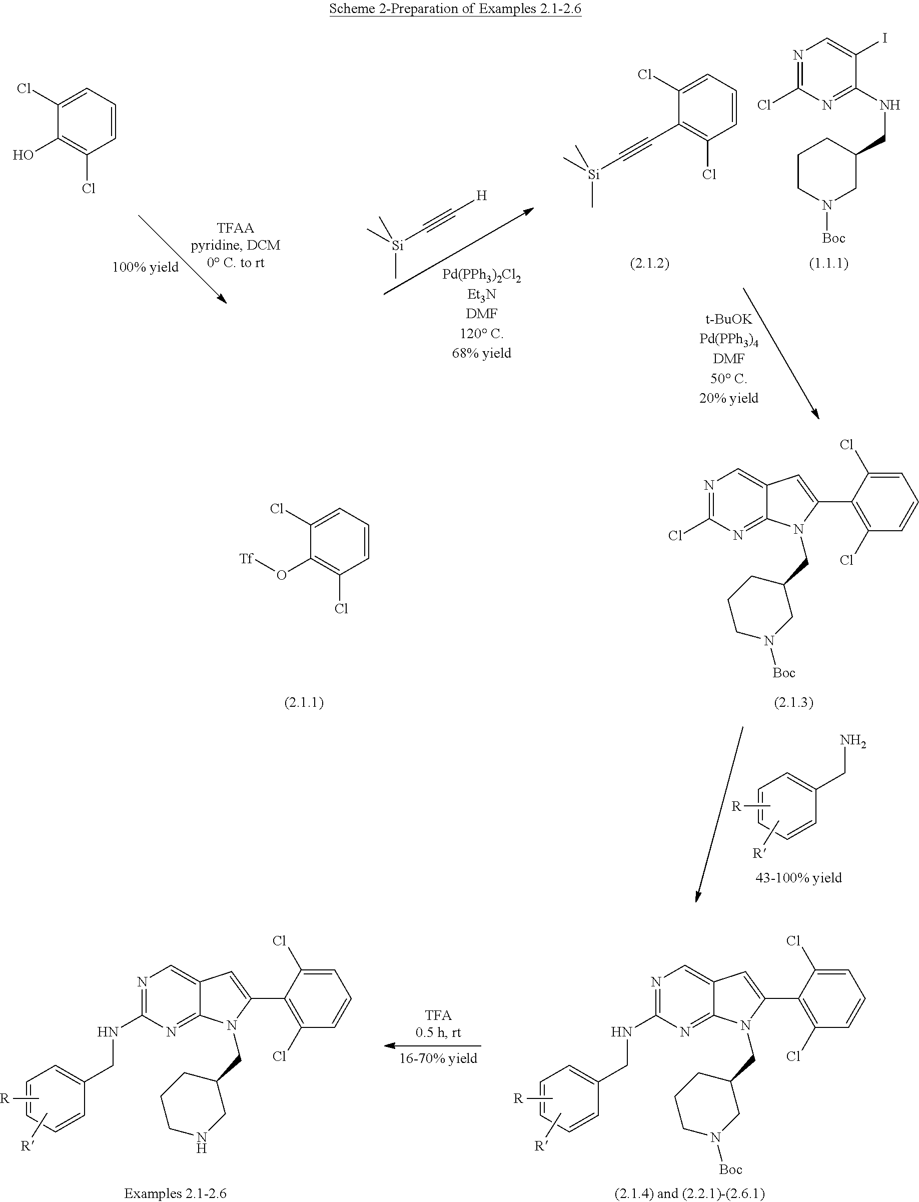

### EXAMPLE 2.1

### (R)-3-((6-(2,6-dichlorophenyl)-7-(piperidin-3-ylmethyl)-7H-pyrrolo[2,3-d]pyrimidin-2-ylamino)methyl)phenol

### (2.1.1) 2,6-Dichlorophenyltrifluoromethanesulphonate

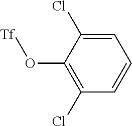

To a solution of 2,6-dichlorophenol (20 g, 123 mmol) in dichloromethane (250 mL) was added pyridine (15.85 mL, 196 mmol). The reaction mixture was cooled to 0° C. and trifluoromethane sulfonic anhydride (29.6 mL, 160 mmol) was added dropwise. The reaction mixture was allowed to warm to room temperature and stirred overnight. The mixture was neutralized with sat. NaHCO₃ solution. The layers were separated. The organic layer was dried (Na₂SO₄), filtered and concentrated in vacuo. To the crude residue heptane was added. The mixture was stirred and precipitation occurred, which was filtered off. The filtrate was concentrated in vacuo. The title compound was obtained in quantitative yield.

### (2.1.2) [(2,6-Dichlorophenyl)ethynyl]trimethylsilane

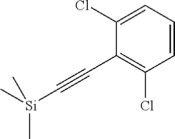

A solution of trimethylsilylacetylene (27.1 mL, 191 mmol), compound (2.1.1) (27.5 g, 127 mmol), bis(triphenylphosphine)palladium(II)chloride (1.78 g, 2.54 mmol) in triethylamine (95 mL) and DMF (475 mL) was heated to 120° C. and stirred overnight. The mixture was cooled to rt and concentrated in vacuo. The residue was dissolved in heptane (600 mL) and stirred for 30 min. The mixture was washed with water (600 mL 2×) and brine (600 mL 1×). The organic layer was dried (Na₂SO₄), filtered and concentrated in vacuo. The crude product was purified with flash chromatography (SiO₂, 100% heptane as mobile phase). The title compound was obtained as yellow oil in 68% yield (21 g, 86 mmol).

### (2.1.3) (S)-tert-butyl 3-((2-chloro-6-(2,6-dichlorophenyl)-7H-pyrrolo[2,3-d]pyrimidin-7-yl)methyl)piperidine-1-carboxylate

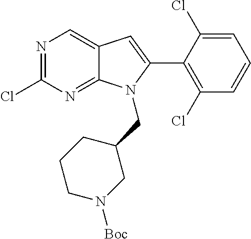

To a solution of compound (1.1.1) (2.3 g, 5.08 mmol) and compound (2.1.2) (1.48 g, 6.1 mmol) in DMF (25 mL) was added potassium tert-butoxide (855 mg, 7.62 mmol) and tetrakis(triphenylphosphine)palladium(0) (294 mg, 0.254 mmol) to give a brown solution. The reaction mixture was heated to 50° C. and stirred overnight. The reaction mixture was poured into a water/EtOAc mixture (1/1). The layers were separated and the organic layer was washed with water (3×), brine (1×), dried (Na₂SO₄), filtered and concentrated in vacuo. The crude product was purified with flash chromatography (SiO₂, heptane/EtOAc; 100% heptane to 50% EtOAc as mobile phase). The title compound was obtained as yellow oil in 20% yield (511 mg, 1.03 mmol). LC-MS: peak at 4.55 min., mass [M+H]: 495.

### (2.1.4) (R)-tert-butyl 3-((6-(2,6-dichlorophenyl)-2-(3-hydroxvbenzylamino)-7H-pyrrolo[2,3-d]pyrimidin-7-yl)methyl)piperidine-1-carboxylate

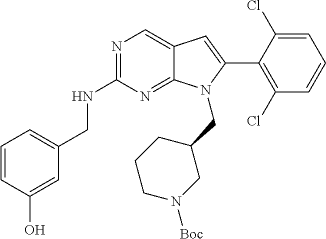

To a solution of compound (2.1.3) (100 mg, 0.20 mmol) in NMP (0.5 mL) was added 3-hydroxybenzylamine (120 mg, 0.97 mmol) and DIEA (351 uL, 2.0 mmol). The mixture was stirred in the microwave for 4 hours at 150° C. and was then diluted with DCM, washed with NH₄Cl solution (2×), followed by water (2×) and brine (1×). The organic layer was dried (Na₂SO₄), filtered and concentrated in vacuo. The crude product was purified with flash chromatography (SiO₂, heptane/EtOAc; 50% EtOAc as mobile phase). The title compound was obtained as a yellow solid in 78% yield (92 mg, 0.16 mmol). LC-MS: peak at 3.64 min., mass [M+H]=582.

### (2.1) (R) 3-{[6-(2,6-Dichloro-phenyl)-7-piperidin-3-ylmethyl-7H-pyrrolo[2,3-d]pyrimidin-2-ylamino]-methyl}-phenol

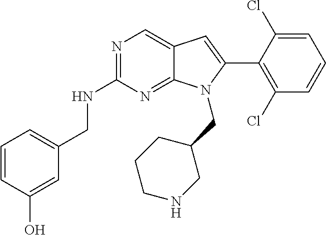

To a solution of compound (2.1.4) (92 mg, 0.16 mmol) in DCM (1 mL) was added TFA (0.5 mL) and the reaction mixture was stirred at room temperature for 30 minutes. The reaction mixture was concentrated in vacuo and the crude product was purified by preparative HPLC (0-50% ACN with TFA, as mobile phase). Product fractions were concentrated and lyophilized to obtain the TFA-salt of the title compound in 70% yield (66 mg). UPLC-MS: peak at 1.64 min., mass [M+H]=482.

### EXAMPLE 2.2

### (R)-6-(2,6-dichlorophenyl)-N-(3,4-difluorobenzyl)-7-(piperidin-3-ylmethyl)-7H-pyrrolo[2,3-d]pyrimidin-2-amine

### (2.2.1) (S)-tert-butyl 3-((6-(2,6-dichlorophenyl)-2-(3,4-difluorobenzylamino)-7H-pyrrolo[2,3-d]pyrimidin-7-yl)methyl)piperidine-1-carboxylate

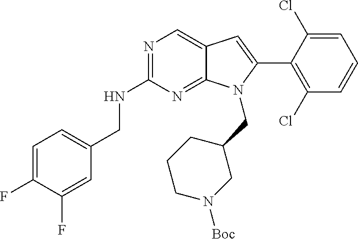

An analogous procedure to that described above to prepare compound 2.1.4 was used except that 3,4-difluorobenzylamine was used instead of 3-hydroxybenzylamine. Yield=43% (35 mg, 0.07 mmol). LC-MS: peak at 3.91 min., mass [M+H]=602.

### (2.2) (R)-6-(2,6-dichlorophenyl)-N-(3,4-difluorobenzyl)-7-(piperidin-3-ylmethyl)-7H-pyrrolo[2,3-d]pyrimidin-2-amine

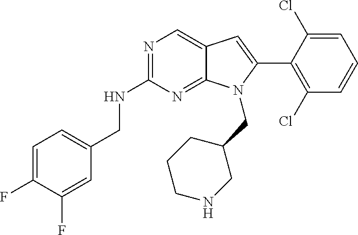

An analogous procedure to that described above to prepare compound 2.1 was used except that compound 2.2.1 was used instead of compound 2.1.4. Yield=44% (18 mg, 0.06 mmol). LC-MS: peak at 2.96 min., mass [M+H]=502.

### EXAMPLE 2.3

### (R)-4-((6-(2,6-dichlorophenyl)-7-(piperidin-3-ylmethyl)-7H-pyrrolo[2,3-d]pyrimidin-2-ylamino)methyl)phenol

### (2.3.1) (S)-tert-butyl 3-((6-(2,6-dichlorophenyl)-2-(4-hydroxybenzylamino)-7H-pyrrolo[2,3-d]pyrimidin-7-yl)methyl)piperidine-1-carboxylate

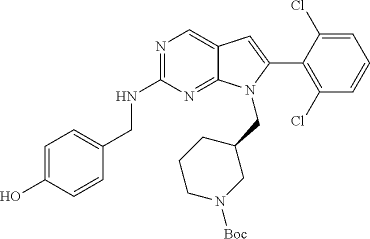

An analogous procedure to that described above to prepare compound 2.1.4 was used except that 4-methoxybenzylamine was used instead of 3-hydroxybenzylamine. Yield=85% (100 mg, 0.17 mmol). LC-MS: peak at 3.68 min., mass [M+H]=582.

### (2.3) (R)-4-((6-(2,6-dichlorophenyl)-7-(piperidin-3-ylmethyl)-7H-pyrrolo[2,3-d]pyrimidin-2-ylamino)methyl)phenol

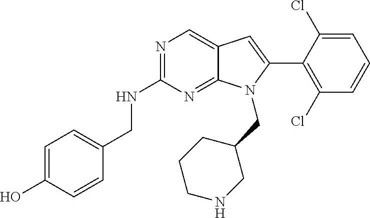

An analogous procedure to that described above to prepare compound 2.1 was used except that compound 2.3.1 was used instead of compound 2.1.4. Yield=16% (16 mg, 0.03 mmol). LC-MS: peak at 2.66 min., mass [M+H]=482.

### EXAMPLE 2.4

### (R)-6-(2,6-dichlorophenyl)-N-(3-fluoro-4-methoxybenzyl)-7-(piperidin-3-ylmethyl)-7H-pyrrolo[2,3-d]pyrimidin-2-amine

### (2.4.1) (S)-tert-butyl 3-((6-(2,6-dichlorophenyl)-2-(3-fluoro-4-methoxybenzylamino)-7H-pyrrolo[2,3-d]pyrimidin-7-yl)methyl)piperidine-1-carboxylate

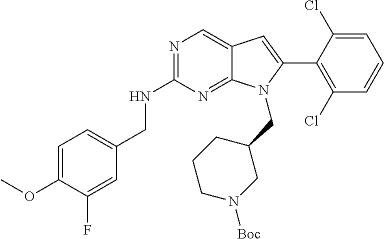

An analogous procedure to that described above to prepare compound 2.1.4 was used except that 3-fluoro-4-methoxybenzylamine was used instead of 3-hydroxybenzylamine. Yield=100% crude (119 mg, 0.19 mmol). LC-MS: peak at 3.90 min., mass [M+H]=614.

### (2.4) (R)-6-(2,6-dichlorophenyl)-N-(3-fluoro-4-methoxybenzyl)-7-(piperidin-3-ylmethyl)-7H-pyrrolo[2,3-d]pyrimidin-2-amine

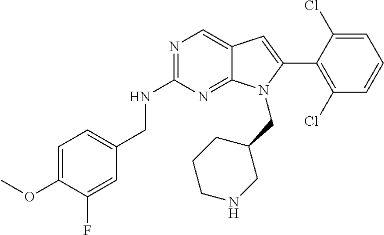

An analogous procedure to that described above to prepare compound 2.1 was used except that compound 2.4.1 was used instead of compound 2.1.4. Yield=33% (20 mg, 0.03 mmol). UPLC-MS: peak at 1.95 min., mass [M+H]=514.

### EXAMPLE 2.5

### (R)-6-(2,6-dichlorophenyl)-N-(3-fluorobenzyl)-7-(piperidin-3-ylmethyl)-7H-pyrrolo[2,3-d]pyrimidin-2-amine

### (2.5.1) (S)-tert-butyl 3-((6-(2,6-dichlorophenyl)-2-(3-fluorobenzylamino)-7H-pyrrolo[2,3-d]pyrimidin-7-yl)methyl)piperidine-1-carboxylate

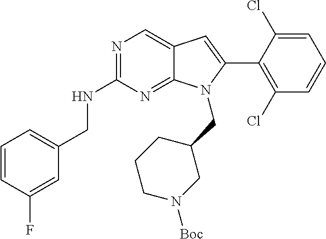

An analogous procedure to that described above to prepare compound (2.1.4) was used, except that 3-fluorobenzylamine was used instead of 3-hydroxybenzylamine. Yield=61% (72 mg, 0.123 mmol). LC-MS: peak at 4.00 min., mass [M+H]=584.

### (2.5) (R)-6-(2,6-dichlorophenyl)-N-(3-fluorobenzyl)-7-(piperidin-3-ylmethyl)-7H-pyrrolo[2,3-d]pyrimidin-2-amine

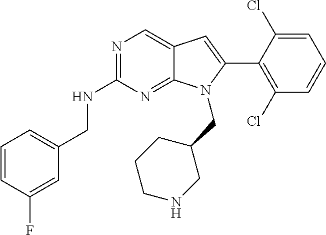

An analogous procedure to that described above to prepare compound 2.1 was used, except that compound (2.5.1) was used instead of compound (2.1.4). Yield=36% (32 mg, 0.04 mmol). UPLC-MS: peak at 2.01 min., mass [M+H]=484.

### EXAMPLE 2.6

### (R)-N-benzyl-6-(2,6-dichlorophenyl)-7-(piperidin-3-ylmethyl)-7H-pyrrolo[2,3-d]pyrimidin-2-amine

### (2.6.1) (S)-tert-butyl 3-((2-(benzylamino)-6-(2,6-dichlorophenyl)-7H-pyrrolo[2,3-d]pyrimidin-7-yl)methyl)piperidine-1-carboxylate

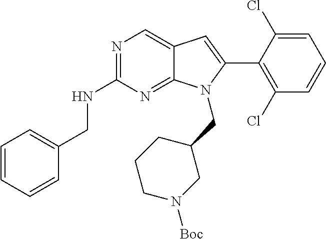

An analogous procedure to that described above to prepare compound (2.1.4) was used, except that benzylamine was used instead of 3-hydroxybenzylamine. Yield=84% (45 mg, 0.08 mmol). MS (ESI): mass [M+H]=566.

### (2.6) (R)—N-benzyl-6-(2,6-dichlorophenyl)-7-(piperidin-3-ylmethyl)-7H-pyrrolo[2,3-d]pyrimidin-2-amine

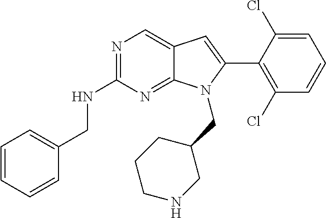

An analogous procedure to that described above to prepare compound 2.1 was used, except that compound (2.6.1) was used instead of compound (2.1.4). Yield=38% (14 mg, 0.03 mmol). UPLC-MS: peak at 2.14 min., mass [M+H]=466.

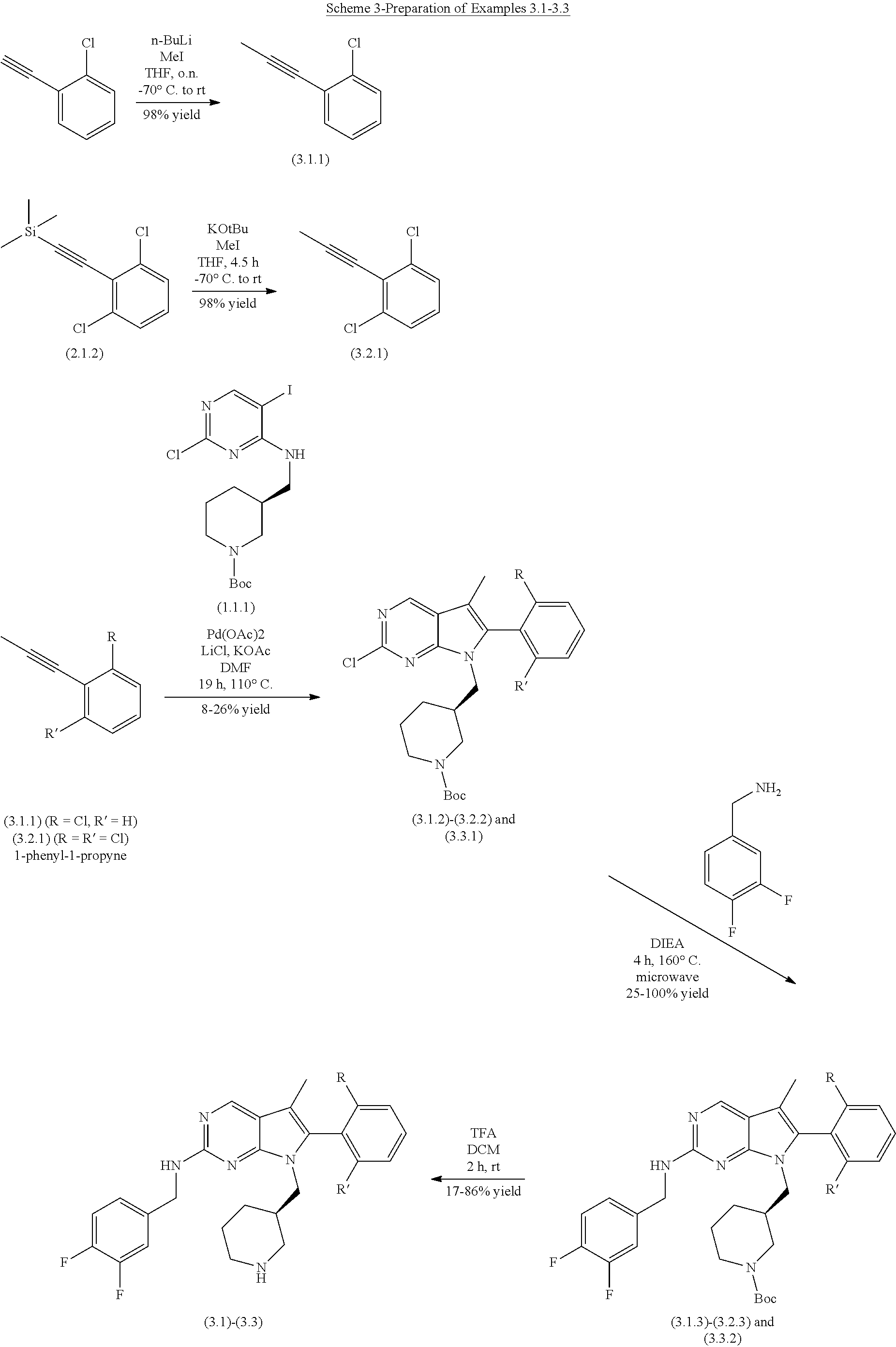

### EXAMPLE 3.1

### (R)-6-(2-chlorophenyl)-N-(3,4-difluorobenzyl)-5-methyl-7-(piperidin-3-ylmethyl)-7H-pyrrolo[2,3-d]pyrimidin-2-amine

### (3.1.1) 1-Chloro-2-(prop-1-ynyl)benzene

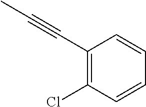

To a stirred solution of 1-chloro-2-ethynylbenzene (0.59 g, 4.32 mmol) in THF (15 mL) at −70° C. was added dropwise a solution of n-butyl lithium 1.6 M in hexane (3.24 mL, 5.18 mmol). After addition the reaction mixture was stirred for 15 minutes at −70° C. Then, a solution of iodomethane (0.54 mL, 8.64 mmol) in THF (5 mL) was added dropwise. The reaction mixture was allowed to warm up to room temperature overnight. Then, the reaction mixture was diluted with EtOAc and washed with brine (1×). The organic layer was dried (Na₂SO₄), filtered and concentrated in vacuo after which the title compound was furnished in 98% yield (0.64 g, 4.25 mmol). LC-MS: UV-peak at 3.96 min., mass [M+H]=no response.

### (3.1.2) (S)-tert-butyl 3-((2-chloro-6-(2-chlorophenyl)-5-methyl-7H-pyrrolo[2,3-d]pyrimidin-7-yl)methyl)piperidine-1-carboxylate

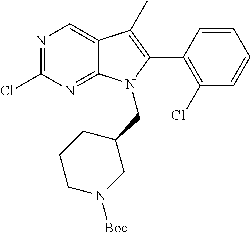

A nitrogen flow was led through a stirred solution of compound (1.1.1) (500 mg, 1.10 mmol) in DMF (8 mL) at room temperature. Subsequently compound (3.1.1) (250 mg, 1.66 mmol), Pd(OAc)₂ (12.4 mg, 0.06 mmol), potassium acetate (217 mg, 2.21 mmol) and lithium chloride (47 mg, 1.10 mmol) were added and the reaction mixture was heated to 110° C. and stirred overnight. The reaction mixture was additionally stirred for 4 days at room temperature and then diluted with EtOAc, washed with water (2×) and brine (1×). The organic layer was dried (Na₂SO₄), filtered and concentrated in vacuo. The crude product was purified by column chromatography (SiO₂, heptane/EtOAc; 100% heptane to 20% EtOAc as mobile phase) and the title compound was obtained as colorless oil in 20% yield (106 mg, 0.22 mmol). LC-MS: peak at 4.59 min., mass [M+H]=475.

### (3.1.3) (R)-tert-butyl 3-((6-(2-chlorophenyl)-2-(3,4-difluorobenzylamino)-5-methyl-7H-pyrrolo[2,3-d]pyrimidin-7-yl)methyl)piperidine-1-carboxylate

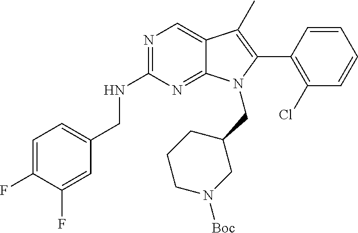

Compound (3.1.2) (106 mg, 0.22 mmol) was dissolved in a mixture of DIEA (0.1 mL) and 3,4-difluorobenzylamine (0.8 mL). Subsequently, the reaction mixture was heated in the microwave for 4 h at 160° C. Then, the reaction mixture was diluted with DCM and washed with saturated NH₄Cl solution (2×) and brine (1×). The organic layer was dried (Na₂SO₄), filtered and concentrated in vacuo. The crude product was purified by column chromatography (SiO₂, heptane/EtOAc; 100% heptane to 30% EtOAc as mobile phase) and the title compound was obtained as colorless oil in 44% yield (57 mg, 0.10 mmol). LC-MS: peak at 4.08 min., mass [M+H]=582.

### (3.1) (R)-6-(2-chlorophenyl)-N-(3,4-difluorobenzyl)-5-methyl-7-(piperidin-3-ylmethyl)-7H-pyrrolo[2,3-d]pyrimidin-2-amine

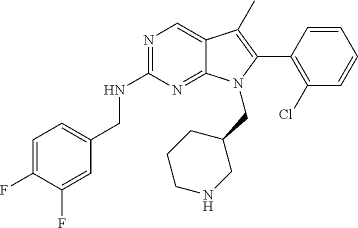

To a solution of compound (3.1.3) (57 mg, 0.10 mmol) in dichloromethane (4 mL) was added TFA (1 mL) at room temperature. Then, the reaction mixture was concentrated in vacuo and crude product was purified by preparative HPLC (0-50% ACN with TFA, as mobile phase). Product fractions were concentrated in vacuo and lyophilized to obtain the TFA-salt of the title compound in 22% yield (12 mg, 0.02 mmol). LC-MS: peak at 3.01 min., mass [M+H]=482.

### EXAMPLE 3.2

### (R)-6-(2,6-dichlorophenyl)-N-(3,4-difluorobenzyl)-5-methyl-7-(piperidin-3-ylmethyl)-7H-pyrrolo[2,3-d]pyrimidin-2-amine

### (3.2.1) 1,3-Dichloro-2-(prop-1-ynyl)benzene

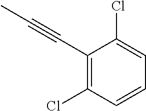

To a stirred solution of compound (2.1.2) (0.97 g, 3.99 mmol) in THF (30 mL) at −70° C. was added potassium tert-butoxide (0.54 g, 4.79 mmol). After addition the reaction mixture was stirred for 45 minutes at −70° C. Then, iodomethane (0.50 mL, 7.98 mmol) was added dropwise. The reaction mixture was allowed to warm up to room temperature. After stirring for 4 h30′ the reaction mixture was diluted with EtOAc and washed with brine (1×). The organic layer was dried (Na₂SO₄), filtered and concentrated in vacuo. The crude product was purified by column chromatography (SiO₂, heptane as mobile phase) after which the title compound was furnished in 66% yield (0.49 g, 2.65 mmol). LC-MS: UV-peak at 4.18 min., mass [M+H]=no response.

### (3.2.2) (S)-tert-butyl 3-((2-chloro-6-(2,6-dichlorophenyl)-5-methyl-7H-pyrrolo[2,3-d]pyrimidin-7-yl)methyl)piperidine-1-carboxylate

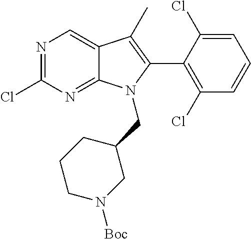

An analogous procedure to that described above to prepare compound 3.1.2 was used except that compound 3.2.1 was used instead of compound 3.1.1. Yield=8% (36 mg, 0.07 mmol). LC-MS: peak at 4.71 min., mass [M+H]=509.

### (3.2.3) (S)-tert-butyl 3-((6-(2,6-dichlorophenyl)-2-(3,4-difluorobenzylamino)-5-methyl-7H-pyrrolo[2,3-d]pyrimidin-7-yl)methyl)piperidine-1-carboxylate

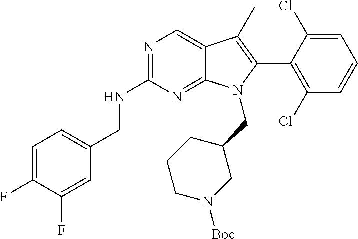

An analogous procedure to that described above to prepare compound 3.1.3 was used except that compound 3.2.2 was used instead of compound 3.1.2. Yield=100% (38 mg, 0.06 mmol). LC-MS: peak at 4.18 min., mass [M+H]=616.

### (3.2) (R)-6-(2,6-dichlorophenyl)-N-(3,4-difluorobenzyl)-5-methyl-7-(piperidin-3-ylmethyl)-7H-pyrrolo[2,3-d]pyrimidin-2-amine

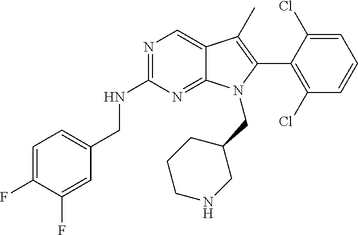

An analogous procedure to that described above to prepare compound 3.1 was used except that compound 3.2.3 was used instead of compound 3.1.3. Yield=17% (7 mg, 0.01 mmol). UPLC-MS: peak at 2.24 min., mass [M+H]=516.

### EXAMPLE 3.3

### (R)—N-(3,4-difluorobenzyl)-5-methyl-6-phenyl-7-(piperidin-3-ylmethyl)-7H-pyrrolo[2,3-d]pyrimidin-2-amine

### (3.3.1) (S)-tert-butyl 3-((2-chloro-5-methyl-6-phenyl-7H-pyrrolo[2,3-d]pyrimidin-7-yl)methyl)piperidine-1-carboxylate

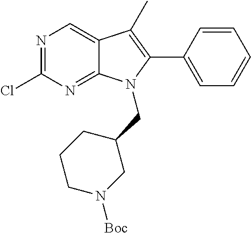

An analogous procedure to that described above to prepare compound 3.1.2 was used except that commercially available 1-phenyl-1-propyne was used instead of compound 3.1.1. Yield=26% (50 mg, 0.11 mmol). LC-MS: peak at 4.50 min., mass [M+H]=441.

### (3.3.2) (S)-tert-butyl 3-((2-(3,4-difluorobenzylamino)-5-methyl-6-phenyl-7H-pyrrolo[2,3-d]pyrimidin-7-yl)methyl)piperidine-1-carboxylate

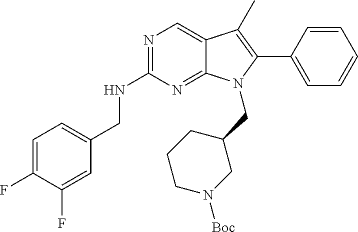

An analogous procedure to that described above to prepare compound 3.1.3 was used except that compound 3.3.1 was used instead of compound 3.1.2. Yield=25% (15 mg, 0.03 mmol). LC-MS: peak at 4.08 min., mass [M+H]=548.

### (3.3) (R)—N-(3,4-difluorobenzyl)-5-methyl-6-phenyl-7-(piperidin-3-ylmethyl)-7H-pyrrolo[2,3-d]pyrimidin-2-amine

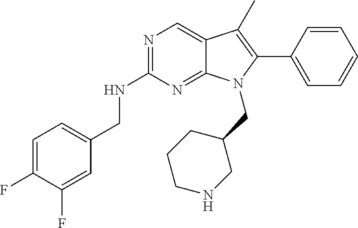

An analogous procedure to that described above to prepare compound 3.1 was used except that compound 3.3.2 was used instead of compound 3.1.3. Yield=86% (13 mg, 0.02 mmol). UPLC-MS: peak at 2.91 min., mass [M+H]=448.

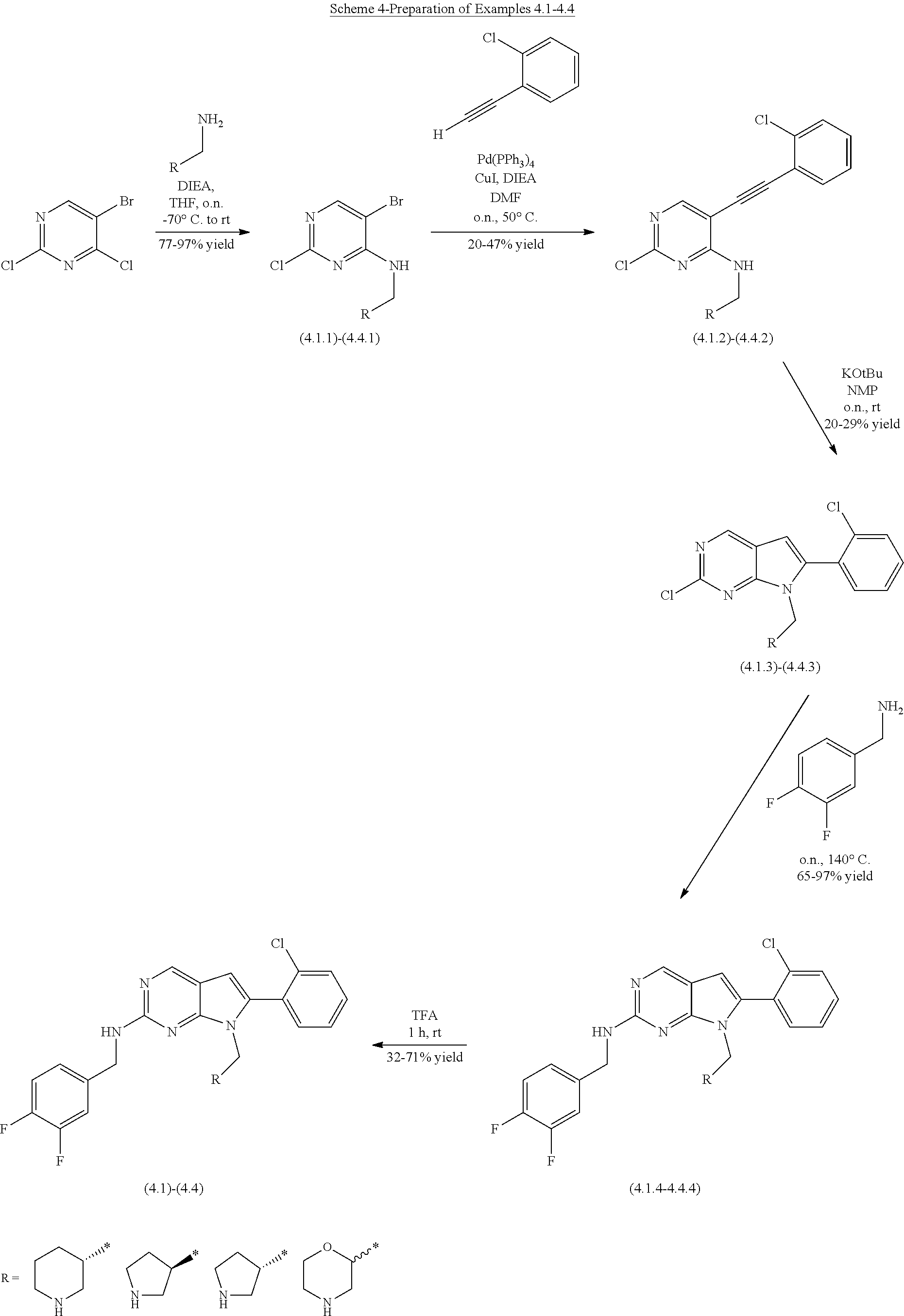

### EXAMPLE 4.1

### (S)-6-(2-chlorophenyl)-N-(3,4-difluorobenzyl)-7-(piperidin-3-ylmethyl)-7H-pyrrolo[2,3-d]pyrimidin-2-amine

### (4.1.1) (R)-tert-butyl 3-((5-bromo-2-chloropyrimidin-4-ylamino)methyl)piperidine-1-carboxylate

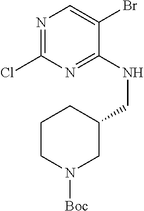

To a stirred solution of 5-bromo-2,4-dichloropyrimidine (0.25 g, 1.097 mmol) in THF (8 mL) at −70° C. was added dropwise a solution of (3R)-3-(aminomethyl)-1-(t-butoxycarbonyl)piperidine (3.0 g, 14 mmol) and DIEA (0.248 mL, 1.426 mmol) in THF (50 mL). The reaction mixture was stirred at −70° C. and then allowed to warm up to room temperature overnight. The reaction mixture was diluted with EtOAc and washed with saturated NH₄Cl solution (1×) and brine (1×). The organic layer was dried (Na₂SO₄), filtered and concentrated in vacuo. The crude product was purified by column chromatography (SiO₂, heptane/EtOAc; 10% to 50% EtOAc as mobile phase) and the title compound was obtained in 97% yield (0.43 g, 1.067 mmol). LC-MS: peak at 4.03 min., mass [M+H]=405.

### (4.1.2) (R)-tert-butyl 3-((2-chloro-5-((2-chlorophenyl)ethynyl)pyrimidin-4-ylamino)methyl)piperidine-1-carboxylate

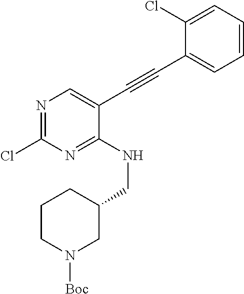

A nitrogen flow was led through a stirred solution of compound (4.1.1) (225 mg, 0.555 mmol) in DMF (3 mL) at room temperature. Subsequently 1-chloro-2-ethynylbenzene (106 mg, 0.776 mmol), DIEA (0.19 mL, 1.109 mmol), copper(I) iodide (7.4 mg, 0.039 mmol) and tetrakis(triphenylphosphine)palladium(0) (64.1 mg, 0.055 mmol) were added and the reaction mixture was stirred at 50° C. overnight. Then, the reaction mixture was diluted with EtOAc and washed with NH₄Cl solution (1×) and brine (1×). The organic layer was dried (Na₂SO₄), filtered and concentrated in vacuo. The crude product was purified by column chromatography (SiO₂, heptane/EtOAc; 100% heptane to 50% EtOAc as mobile phase) and the title compound was obtained in 47% yield (120 mg, 0.26 mmol). MS (ESI): mass [M+H]=461.

### (4.1.3) (R)-tert-butyl 3-((2-chloro-6-(2-chlorophenyl)-7H-pyrrolo[2,3-d]pyrimidin-7-yl)methyl)piperidine-1-carboxylate

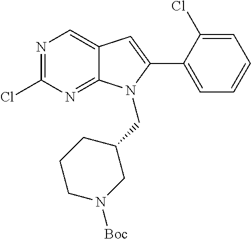

A solution of compound (4.1.2) (120 mg, 0.26 mmol) in NMP (2 mL) was added to a stirred suspension of potassium tert-butoxide (58 mg, 0.52 mmol) in NMP (1 mL) at room temperature. Subsequently the reaction mixture was stirred at room temperature overnight. Then, the reaction mixture was diluted with EtOAc and washed with water (1×) and brine (1×). The organic layer was dried (Na₂SO₄), filtered and concentrated in vacuo. The crude product was purified by column chromatography (SiO₂, heptane/EtOAc; 100% heptane to 40% EtOAc as mobile phase) to give the title compound in 23% yield (28 mg, 0.061 mmol). MS (ESI): mass [M+H]=461.

### (4.1.4) (R)-tert-butyl 3-((6-(2-chlorophenyl)-2-(3,4-difluorobenzylamino)-7H-pyrrolo[2,3-d]pyrimidin-7-yl)methyl)piperidine-1-carboxylate

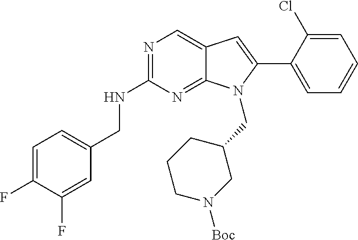

3,4-difluorobenzylamine (0.36 mL, 3.03 mmol) was added to compound (4.1.3) (28 mg, 0.061 mmol) and the reaction mixture stirred at 140° C. overnight. Then, the reaction mixture was diluted with EtOAc and washed with saturated NH₄Cl solution (1×) and brine (1×). The organic layer was dried (Na₂SO₄), filtered and concentrated in vacuo. The crude product was purified by column chromatography (SiO₂, heptane/EtOAc; 100% heptane to 50% EtOAc as mobile phase) and the title compound was obtained in 69% yield (24 mg, 0.042 mmol).

### (4.1) (S)-6-(2-chlorophenyl)-N-(3,4-difluorobenzyl)-7-(piperidin-3-ylmethyl)-7H-pyrrolo[2,3-d]pyrimidin-2-amine

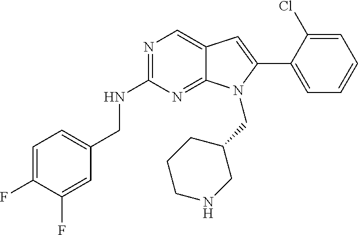

To a stirred solution of compound 4.1.4 (24 mg, 0.042 mmol) in DCM (0.6 mL) was added TFA (0.6 mL). The reaction mixture was stirred at room temperature for 1 h and was subsequently concentrated in vacuo. The crude product was purified by preparative HPLC (0-50% ACN with TFA, as mobile phase). Product fractions were concentrated and lyophilized to obtain the TFA-salt of the title compound in 32% yield (8 mg, 0.014 mmol). UPLC-MS: peak at 1.98 min., mass [M+H]=468.

### EXAMPLE 4.2

### (R)-6-(2-chlorophenyl)-N-(3,4-difluorobenzyl)-7-(pyrrolidin-3-ylmethyl)-7H-pyrrolo[2,3-d]pyrimidin-2-amine

### (4.2.1) (S)-tert-butyl 3-((5-bromo-2-chloropyrimidin-4-ylamino)methyl)pyrrolidine-1-carboxylate

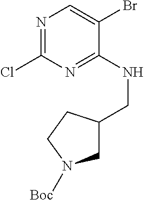

An analogous procedure to that described above to prepare compound 4.1.1 was used except that (3S)-(aminomethyl)-1-(t-butoxycarbonyl)pyrrolidine was used instead of (3R)-(aminomethyl)-1-(t-butoxycarbonyl)piperidine. Yield=77% (0.33 gram, 0.85 mmol). LC-MS: peak at 3.82 min., mass [M+H]=391, 393.

### (4.2.2) (S)-tert-butyl 3-((2-chloro-5-((2-chlorophenyl)ethynyl)pyrimidin-4-ylamino)methyl)pyrrolidine-1-carboxylate

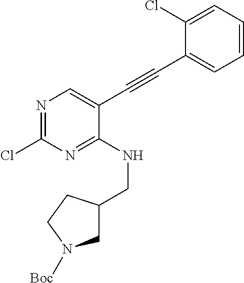

An analogous procedure to that described above to prepare compound 4.1.2 was used except that compound 4.2.1 was used in place of compound 4.1.1. Yield=40% (78 mg, 0.17 mmol). LC-MS: peak at 4.69 min., mass [M+H]=447.

### (4.2.3) (S)-tert-butyl 3-((2-chloro-6-(2-chlorophenyl)-7H-pyrrolo[2,3-d]pyrimidin-7-yl)methyl)pyrrolidine-1-carboxylate

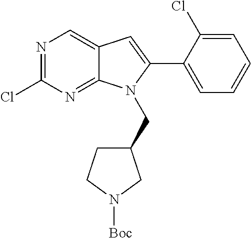

An analogous procedure to that described above to prepare compound 4.1.3 was used except that compound 4.2.2 was used in place of compound 4.1.2. Yield=26% (21 mg, 0.046 mmol). MS (ESI): mass [M+H]=447.

### (4.2.4) (S)-tert-butyl 3-((6-(2-chlorophenyl)-2-(3,4-difluorobenzylamino)-7H-pyrrolo[2,3-d]pyrimidin-7-yl)methyl)pyrrolidine-1-carboxylate

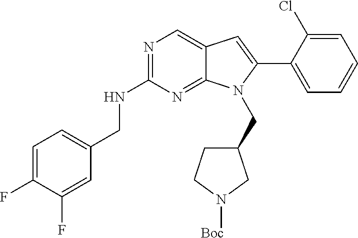

An analogous procedure to that described above to prepare compound 4.1.4 was used except that compound 4.2.3 was used in place of compound 4.1.3. Yield=88% (22 mg, 0.040 mmol). MS (ESI): mass [M+H]=554.

### (4.2) (R)-6-(2-chlorophenyl)-N-(3,4-difluorobenzyl)-7-(pyrrolidin-3-ylmethyl)-7H-pyrrolo[2,3-d]pyrimidin-2-amine

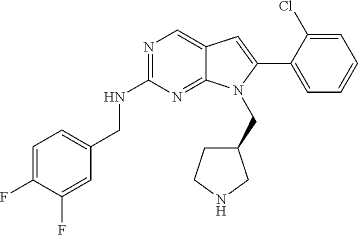

An analogous procedure to that described above to prepare compound 4.1 was used except that compound 4.2.4 was used in place of compound 4.1.4. Yield=64% (15 mg, 0.026 mmol). UPLC-MS: peak at 1.91 min., mass [M+H]=454.

### EXAMPLE 4.3

### (S)-6-(2-chlorophenyl)-N-(3,4-difluorobenzyl)-7-(pyrrolidin-3-ylmethyl)-7H-pyrrolo[2,3-d]pyrimidin-2-amine

### (4.3.1) (R)-tert-butyl 3-((5-bromo-2-chloropyrimidin-4-ylamino)methyl)pyrrolidine-1-carboxylate

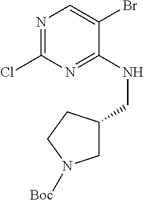

An analogous procedure to that described above to prepare compound 4.1.1 was used except that (3R)-(aminomethyl)-1-(t-butoxycarbonyl)pyrrolidine was used instead of (3R)-(aminomethyl)-1-(t-butoxycarbonyl)piperidine. Yield=91% (0.39 gram, 1.00 mmol). LC-MS: peak at 3.80 min., mass [M+H]=391, 393.

### (4.3.2) (R)-tert-butyl 3-((2-chloro-5-((2-chlorophenyl)ethynyl)pyrimidin-4-ylamino)methyl)pyrrolidine-1-carboxylate

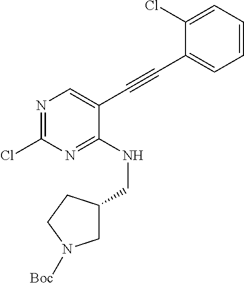

An analogous procedure to that described above to prepare compound 4.1.2 was used except that compound 4.3.1 was used in place of compound 4.1.1. Yield=20% (46 mg, 0.10 mmol). MS (ESI): mass [M+H]=447.

### (4.3.3) (R)-tert-butyl 3-((2-chloro-6-(2-chlorophenyl)-7H-pyrrolo[2,3-d]pyrimidin-7-yl)methyl)pyrrolidine-1-carboxylate

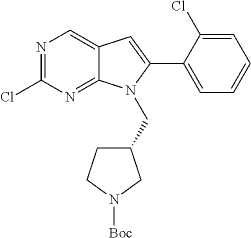

An analogous procedure to that described above to prepare compound 4.1.3 was used except that compound 4.3.2 was used in place of compound 4.1.2. Yield=21% (10 mg, 0.022 mmol). MS (ESI): mass [M+H]=447.

### (4.3.4) (R)-tert-butyl 3-((6-(2-chlorophenyl)-2-(3,4-difluorobenzylamino)-7H-pyrrolo[2,3-d]pyrimidin-7-yl)methyl)pyrrolidine-1-carboxylate

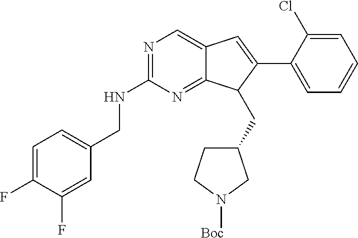

An analogous procedure to that described above to prepare compound 4.1.3 was used except that compound 4.3.3 was used in place of compound 4.1.3. Yield=65% (8 mg, 0.014 mmol). MS (ESI): mass [M+H]=554.

### (4.3) (S)-6-(2-chlorophenyl)-N-(3,4-difluorobenzyl)-7-(pyrrolidin-3-ylmethyl)-7H-pyrrolo[2,3-d]pyrimidin-2-amine

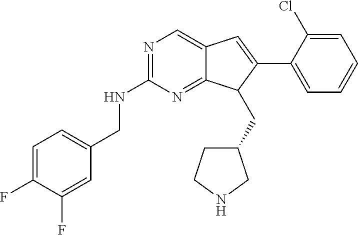

An analogous procedure to that described above to prepare compound 4.1 was used except that compound 4.3.4 was used in place of compound 4.1.4. Yield=71% (6 mg, 0.010 mmol). UPLC-MS: peak at 1.89 min., mass [M+H]=454.

### EXAMPLE 4.4

### 6-(2-Chlorophenyl)-N-(3,4-difluorobenzyl)-7-(morpholin-2-ylmethyl)-7H-pyrrolo[2,3-d]pyrimidin-2-amine

### (4.4.1) Tert-butyl 2-((5-bromo-2-chloropyrimidin-4-ylamino)methyl)morpholine-4-carboxylate

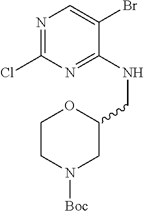

An analogous procedure to that described above to prepare compound 4.1.1 was used except that tert-butyl 2-(aminomethyl)morpholine-4-carboxylate was used instead of (3R)-(aminomethyl)-1-(t-butoxycarbonyl)piperidine. Yield=88% (0.39 gram, 0.97 mmol). LC-MS: peak at 3.70 min., mass [M+H]=407, 409.

### (4.4.2) Tert-butyl 2-((2-chloro-5-((2-chlorophenyl)ethynyl)pyrimidin-4-ylamino)methyl)-morpholine-4-carboxylate

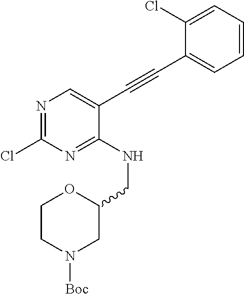

An analogous procedure to that described above to prepare compound 4.1.2 was used except that compound 4.4.1 was used in place of compound 4.1.1. Yield=44% (0.10 gram, 0.22 mmol). LC-MS: peak at 4.70 min., mass [M+H]=463.

### (4.4.3) Tert-butyl 2-((2-chloro-6-(2-chlorophenyl)-7H-pyrrolo[2,3-d]pyrimidin-7-yl)methyl)-morpholine-4-carboxylate

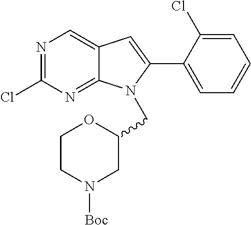

An analogous procedure to that described above to prepare compound 4.1.3 was used except that compound 4.4.2 was used in place of compound 4.1.2. Yield=29% (29 mg, 0.062 mmol). MS (ESI): mass [M+H]=463.

### (4.4.4) Tert-butyl 2-((6-(2-chlorophenyl)-2-(3,4-difluorobenzylamino)-7H-pyrrolo[2,3-d]pyrimidin-7-yl)methyl)morpholine-4-carboxylate

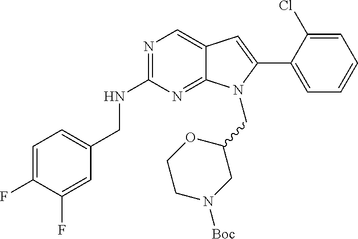

An analogous procedure to that described above to prepare compound 4.1.4 was used except that compound 4.4.3 was used in place of compound 4.1.3. Yield=97% (34 mg, 0.060 mmol). MS (ESI): mass [M+H]=570.

### (4.4) 6-(2-chlorophenyl)-N-(3,4-difluorobenzyl)-7-(morpholin-2-ylmethyl)-7H-pyrrolo[2,3-d]pyrimidin-2-amine

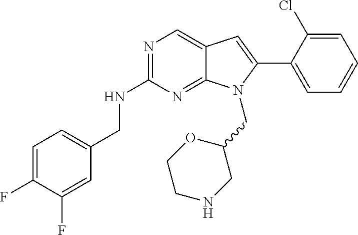

An analogous procedure to that described above to prepare compound 4.1 was used except that compound 4.4.4 was used in place of compound 4.1.4. Yield=64% (22 mg, 0.038 mmol). UPLC-MS: peak at 1.94 min., mass [M+H]=470.

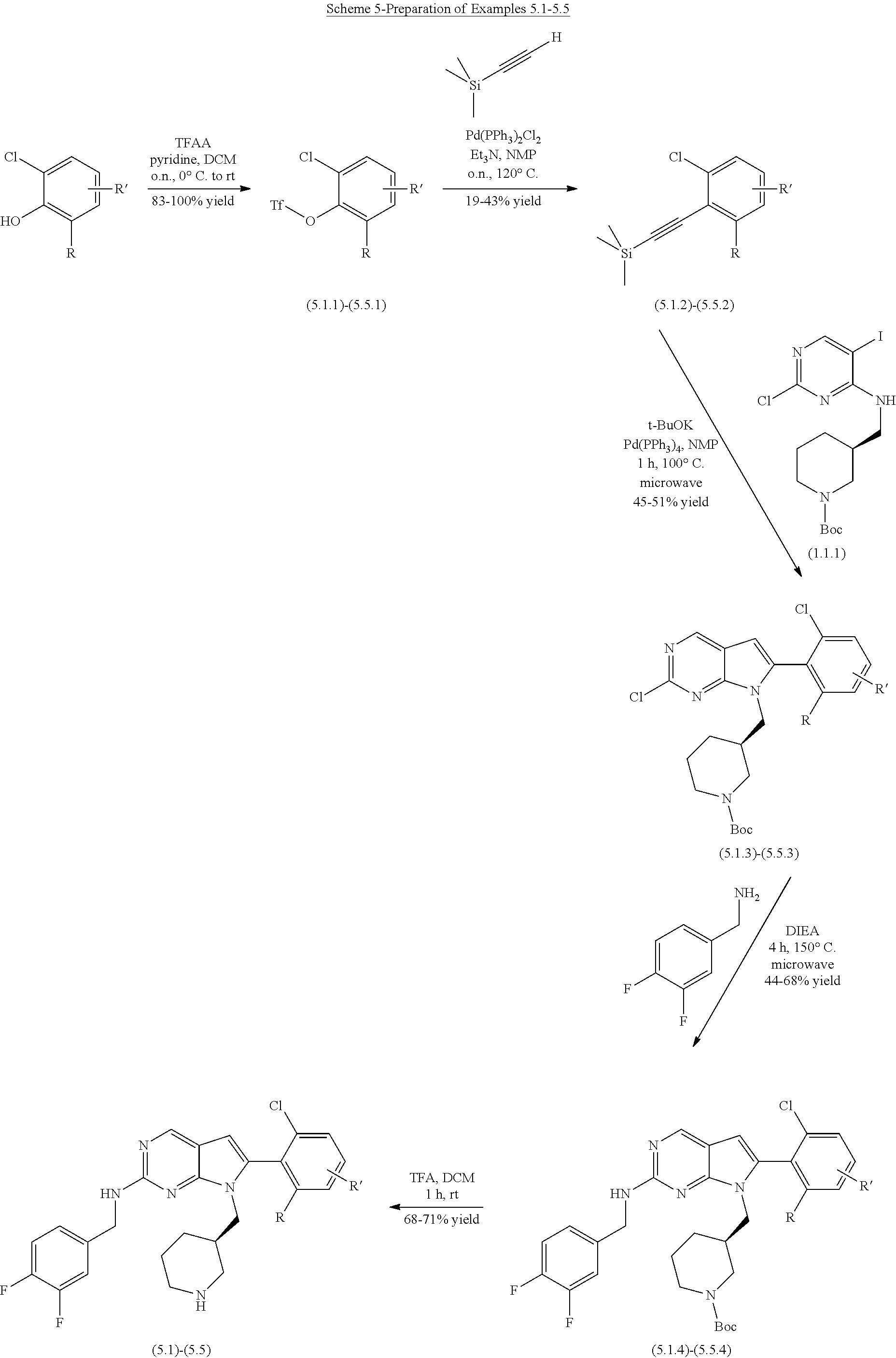

### EXAMPLE 5.1

### (R)-6-(2-chloro-4-methylphenyl)-N-(3,4-difluorobenzyl)-7-(piperidin-3-ylmethyl)-7H-pyrrolo[2,3-d]pyrimidin-2-amine

### (5.1.1) 2-Chloro-4-methylphenyltrifluoromethanesulfonate

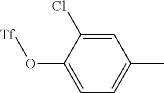

To a solution of 2-chloro-4-methylphenol (0.83 mL, 7.0 mmol) in dichloromethane (20 mL) was added pyridine (0.91 mL, 11.2 mmol). The reaction mixture was cooled to 0° C. and trifluoromethane sulfonic anhydride (1.54 mL, 9.1 mmol) was added dropwise. The reaction mixture was allowed to warm to room temperature and stirred overnight. The mixture was neutralized with sat. NaHCO₃ solution. The layers were separated and the organic layer was dried (Na₂SO₄), filtered and concentrated in vacuo. The residue was coevaporated with toluene to obtain the title compound in quantitative yield.

### (5.1.2) [(2-chloro-4-methylphenyl)ethynyl]trimethylsilane

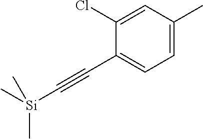

A solution of trimethylsilylacetylene (1.46 mL, 10.3 mmol), compound (5.1.1) (1.88 g, 6.85 mmol), tetrakis(triphenylphosphine)palladium(0) (158 mg, 0.14 mmol) in triethylamine (4.77 mL, 34.2 mmol) and NMP (30 mL) was heated to 120° C. and stirred overnight. The mixture was cooled to rt, diluted with EtOAc and washed with water (1×) and brine (1×). The organic layer was dried (Na₂SO₄), filtered and concentrated in vacuo. The crude product was purified by column chromatography (SiO₂, heptane as mobile phase) and the title compound was obtained in 19% yield (0.28 g, 1.28 mmol).

### (5.1.3) (S)-tert-butyl 3-((2-chloro-6-(2-chloro-4-methylphenyl)-7H-pyrrolo[2,3-d]pyrimidin-7-yl)methyl)piperidine-1-carboxylate

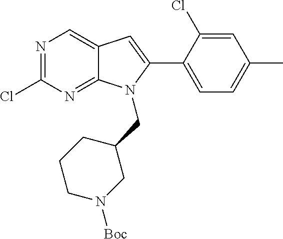

Nitrogen was led through a solution of compound (1.1.1) (0.15 g, 0.33 mmol) and compound (5.1.2) (89 mg, 0.40 mmol) in DMF (2 mL). Potassium tert-butoxide (55.8 mg, 0.50 mmol) and tetrakis(triphenylphosphine)palladium(0) (19 mg, 0.017 mmol) were added and the reaction mixture was heated in the microwave for 1 h at 100° C. The reaction mixture was diluted with EtOAc and washed with water (1×) and brine (1×). The organic layer was dried (Na₂SO₄), filtered and concentrated in vacuo. The crude product was purified by column chromatography (SiO₂, heptane/EtOAc; 100% heptane to 40% EtOAc as mobile phase). The title compound was obtained in 45% yield (71 mg, 0.15 mmol). MS (ESI): mass [M+H]=475.

### (5.1.4) (S)-tert-butyl 3-((6-(2-chloro-4-methylphenyl)-2-(3,4-difluorobenzylamino)-7H-pyrrolo[2,3-d]pyrimidin-7-yl)methyl)piperidine-1-carboxylate

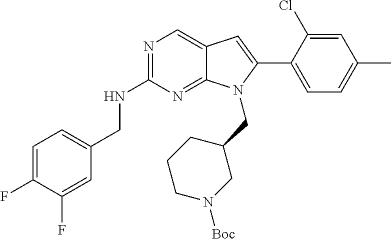

To a solution of compound (5.1.3) (71 mg, 0.15 mmol) in NMP (0.7 mL) were added 3,4-difluorobenzylamine (0.18 mL, 1.49 mmol) and DIEA (52 uL, 0.30 mmol). The mixture was heated in the microwave for 4 hours at 150° C. The reaction mixture was then diluted with EtOAc and washed with NH₄Cl solution (1×) and brine (1×). The organic layer was dried (Na₂SO₄), filtered and concentrated in vacuo. The crude product was purified by column chromatography (SiO₂, heptane/EtOAc; 100% heptane to 50% EtOAc as mobile phase). The title compound was obtained in 44% yield (38 mg, 0.065 mmol). MS (ESI): mass [M+H]=582.

### (5.1) (R)-6-(2-chloro-4-methylphenyl)-N-(3,4-difluorobenzyl)-7-(piperidin-3-ylmethyl)-7H-pyrrolo[2,3-d]pyrimidin-2-amine

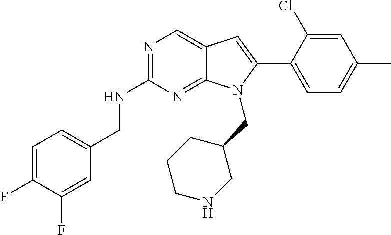

To a stirred solution of compound (5.1.4) (38 mg, 0.065 mmol) in DCM (1 mL) was added TFA (1.0 mL). The reaction mixture was stirred at room temperature for 1 h and was subsequently concentrated in vacuo. The crude product was purified by preparative HPLC (0-50% ACN with TFA, as mobile phase). Product fractions were concentrated and lyophilized to obtain the TFA-salt of the title compound in 69% yield (27 mg, 0.045 mmol). MS (ESI): mass [M+H]=482.

### EXAMPLE 5.2

### (R)-6-(2-chloro-4-methoxyphenyl)-N-(3,4-difluorobenzyl)-7-(piperidin-3-ylmethyl)-7H-pyrrolo[2,3-d]pyrimidin-2-amine

### (5.2.1) 2-Chloro-4-methoxyphenyltrifluoromethanesulfonate

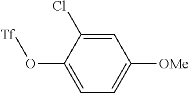

An analogous procedure to that described above to prepare compound 5.1.1 was used except that 2-chloro-4-methoxyphenol was used in place of 2-chloro-4-methylphenol. Yield=97% (1.75 gram, 5.93 mmol).

### (5.2.2) [(2-chloro-4-methoxyphenyl)ethynyl]trimethylsilane

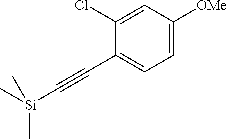

Nitrogen was led through a solution of compound (5.2.1) (0.65 gram, 2.24 mmol) and triethylamine (3.12 mL, 22.4 mmol) in NMP (11 mL). Trimethylsilylacetylene (1.59 mL, 11.2 mmol) and bis(triphenylphospine)palladium(II) chloride (31 mg, 0.045 mmol) were added. The reaction mixture was heated to 120° C. and stirred overnight. The mixture was cooled to rt, diluted with EtOAc and washed with water (1×) and brine (1×). The organic layer was dried (Na₂SO₄), filtered and concentrated in vacuo. The crude product was purified by column chromatography (SiO₂, heptane as mobile phase) and the title compound was obtained in 43% yield (0.23 g, 0.96 mmol).

### (5.2.3) (S)-tert-butyl 3-((2-chloro-6-(2-chloro-4-methoxyphenyl)-7H-pyrrolo[2,3-d]pyrimidin-7-yl)methyl)piperidine-1-carboxylate

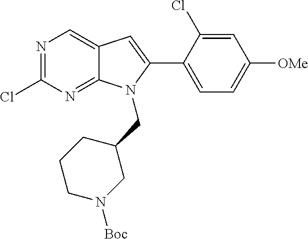

Nitrogen was led through a solution of compound (1.1.1) (0.15 g, 0.33 mmol) and compound (5.2.2) (95 mg, 0.40 mmol) in NMP (2 mL). Potassium tert-butoxide (55.8 mg, 0.50 mmol) and tetrakis(triphenylphosphine)palladium(0) (19 mg, 0.017 mmol) were added and the reaction mixture was heated in the microwave for 3 h at 100° C. An additional amount of compound (5.2.2) (20 mg, 0.084 mmol) and tetrakis(triphenylphosphine)palladium(0) (5 mg, 0.004 mmol) was added and the reaction mixture was heated in the microwave for 1 h at 100° C. The reaction mixture was diluted with EtOAc and washed with water (1×) and brine (1×). The organic layer was dried (Na₂SO₄), filtered and concentrated in vacuo. The crude product was purified by column chromatography (SiO₂, heptane/EtOAc; 100% heptane to 40% EtOAc as mobile phase). The title compound was obtained in 45% yield (71 mg, 0.15 mmol). MS (ESI): mass [M+H]=491.

### (5.2.4) (S)-tert-butyl 3-((6-(2-chloro-4-methoxyphenyl)-2-(3,4-difluorobenzylamino)-7H-pyrrolo[2,3-d]pyrimidin-7-yl)methyl)piperidine-1-carboxylate

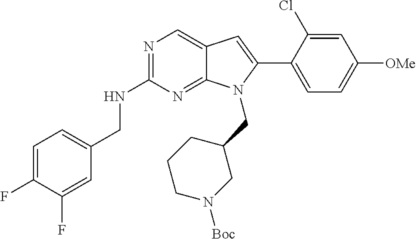

An analogous procedure to that described above to prepare compound 5.1.4 was used except that compound 5.2.3 was used in place of compound 5.1.3. Yield=68% (49 mg, 0.082 mmol). MS (ESI): mass [M+H]=598.

### (5.2) (R)-6-(2-chloro-4-methoxyphenyl)-N-(3,4-difluorobenzyl)-7-(piperidin-3-ylmethyl)-7H-pyrrolo[2,3-d]pyrimidin-2-amine

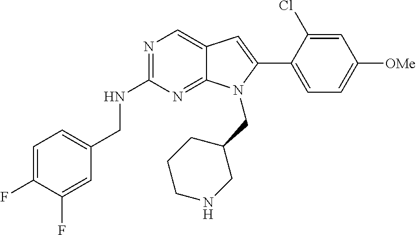

An analogous procedure to that described above to prepare compound 5.1 was used except that compound 5.2.4 was used in place of compound 5.1.4. Yield=71% (15 mg, 0.024 mmol). UPLC-MS: peak at 2.22 min., mass [M+H]=498.

### EXAMPLE 5.3

### (R)-3-chloro-4-(2-(3,4-difluorobenzylamino)-7-(piperidin-3-ylmethyl)-7H-pyrrolo[2,3-d]pyrimidin-6-yl)phenol

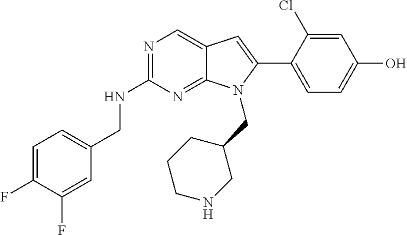

To a stirred solution of compound 5.3 (30 mg, 0.050 mmol) in DCM (3 mL) was added boron trifluoride-methyl sulfide complex (8 uL). The reaction mixture was stirred at room temperature overnight. An additional amount of boron trifluoride-methyl sulfide complex was added and the reaction mixture was refluxed for 4 h. Boron tribromide (40 uL, 0.42 mmol) was added and the reaction mixture was stirred at room temperature for 4 h. Subsequently, the reaction mixture was concentrated in vacuo and the crude product was purified by preparative HPLC (0-40% ACN with TFA, as mobile phase). Product fractions were concentrated and lyophilized to obtain the TFA-salt of the title compound in 72% yield (22 mg, 0.036 mmol). UPLC-MS: peak at 1.96 min., mass [M+H]=484.

### EXAMPLE 5.4

### (R)-6-(2,4-dichlorophenyl)-N-(3,4-difluorobenzyl)-7-(piperidin-3-ylmethyl)-7H-pyrrolo[2,3-d]pyrimidin-2-amine

### (5.4.1) 2,4-Dichlorophenyltrifluoromethanesulfonate

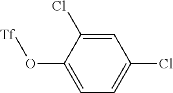

An analogous procedure to that described above to prepare compound 5.1.1 was used except that 2,4-dichlorophenol was used in place of 2-chloro-4-methylphenol. Yield=97% (1.75 gram, 5.93 mmol).

### (5.4.2) [(2,4-dichlorophenyl)ethynyl]trimethylsilane

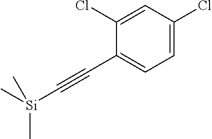

An analogous procedure to that described above to prepare compound 5.2.2 was used except that compound 5.4.1 was used in place of compound 5.2.1. Yield=36% (0.52 gram, 2.14 mmol).

### (5.4.3) (S)-tert-butyl 3-((2-chloro-6-(2,4-dichlorophenyl)-7H-pyrrolo[2,3-d]pyrimidin-7-yl)methyl)piperidine-1-carboxylate

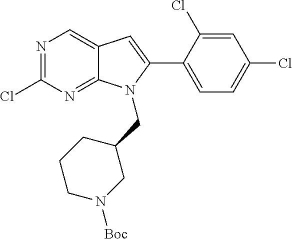

An analogous procedure to that described above to prepare compound 5.1.3 was used except that compound 5.4.2 was used in place of compound 5.1.2. Yield=51% (83 mg, 0.17 mmol).

### (5.4.4) (S)-tert-butyl 3-((6-(2,4-dichlorophenyl)-2-(3,4-difluorobenzylamino)-7H-pyrrolo[2,3-d]pyrimidin-7-yl)methyl)piperidine-1-carboxylate

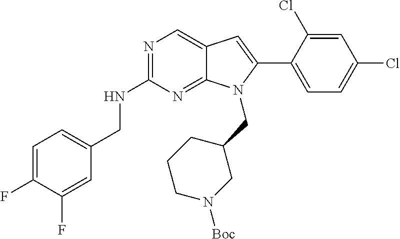

An analogous procedure to that described above to prepare compound 5.1.4 was used except that compound 5.4.3 was used in place of compound 5.1.3. Yield=54% (54 mg, 0.090 mmol). MS (ESI): mass [M+H]=602.

### (5.4) (R)-6-(2,4-dichlorophenyl)-N-(3,4-difluorobenzyl)-7-(piperidin-3-ylmethyl)-7H-pyrrolo-[2,3-d]pyrimidin-2-amine

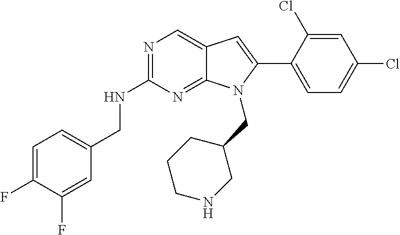

An analogous procedure to that described above to prepare compound 5.1 was used except that compound 5.4.4 was used in place of compound 5.1.4. Yield=68% (38 mg, 0.061 mmol). UPLC-MS: peak at 2.36 min., mass [M+H]=502.

### EXAMPLE 5.5

### (R)-6-(2,6-dichloro-4-fluorophenyl)-N-(3,4-difluorobenzyl)-7-(piperidin-3-ylmethyl)-7H-pyrrolo[2,3-d]pyrimidin-2-amine

### (5.5.1) 2,6-Dichloro-4-fluorophenyl trifluoromethanesulfonate

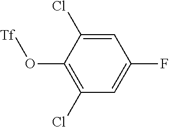

An analogous procedure to that described above to prepare compound (5.1.1) was used, except that 2,6-dichloro-4-fluorophenol was used instead of 2-chloro-4-methylphenol. Yield=94% (1.33 gram, 4.25 mmol).

### (5.5.2) [(2,6-Dichloro-4-fluorophenyl)ethynyl]trimethylsilane

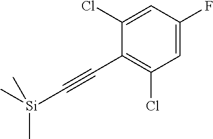

An analogous procedure to that described above to prepare compound (5.2.2) was used, except that compound (5.5.1) was used instead of compound (5.2.1). Yield=52% (0.38 gram, 1.46 mmol).

### (5.5.3) (S)-tert-butyl 3-((2-chloro-6-(2,6-dichloro-4-fluorophenyl)-7H-pyrrolo[2,3-d]pyrimidin-7-yl)methyl)piperidine-1-carboxylate

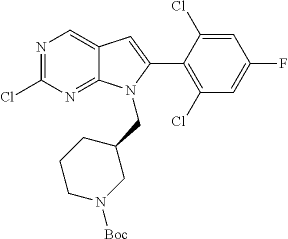

An analogous procedure to that described above to prepare compound (5.1.3) was used, except that compound (5.5.2) was used instead of compound (5.1.2). Yield=34% (62 mg, 0.12 mmol). LC-MS: peak at 5.01 min., mass [M+H]=513.

### (5.5.4) (S)-tert-butyl 3-((6-(2,6-dichloro-4-fluorophenyl)-2-(3,4-difluorobenzylamino)-7H-pyrrolo[2,3-d]pyrimidin-7-yl)methyl)piperidine-1-carboxylate

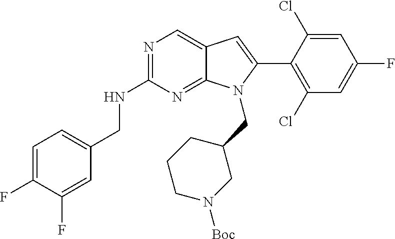

An analogous procedure to that described above to prepare compound (5.1.4) was used, except that compound (5.5.3) was used instead of compound (5.1.3). Yield=25% (22 mg, 0.03 mmol). LC-MS: peak at 4.26 min., mass [M+H]=620.

### (5.5) (R)-6-(2,6-dichloro-4-fluorophenyl)-N-(3,4-difluorobenzyl)-7-(piperidin-3-ylmethyl)-7H-pyrrolo[2,3-d]pyrimidin-2-amine

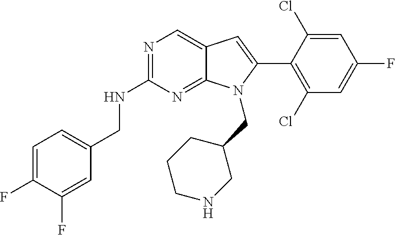

An analogous procedure to that described above to prepare compound 5.1 was used, except that compound (5.5.4) was used in place of compound (5.1.4). Yield=14% (5 mg, 0.009 mmol). UPLC-MS: peak at 2.71 min., mass [M+H]=520

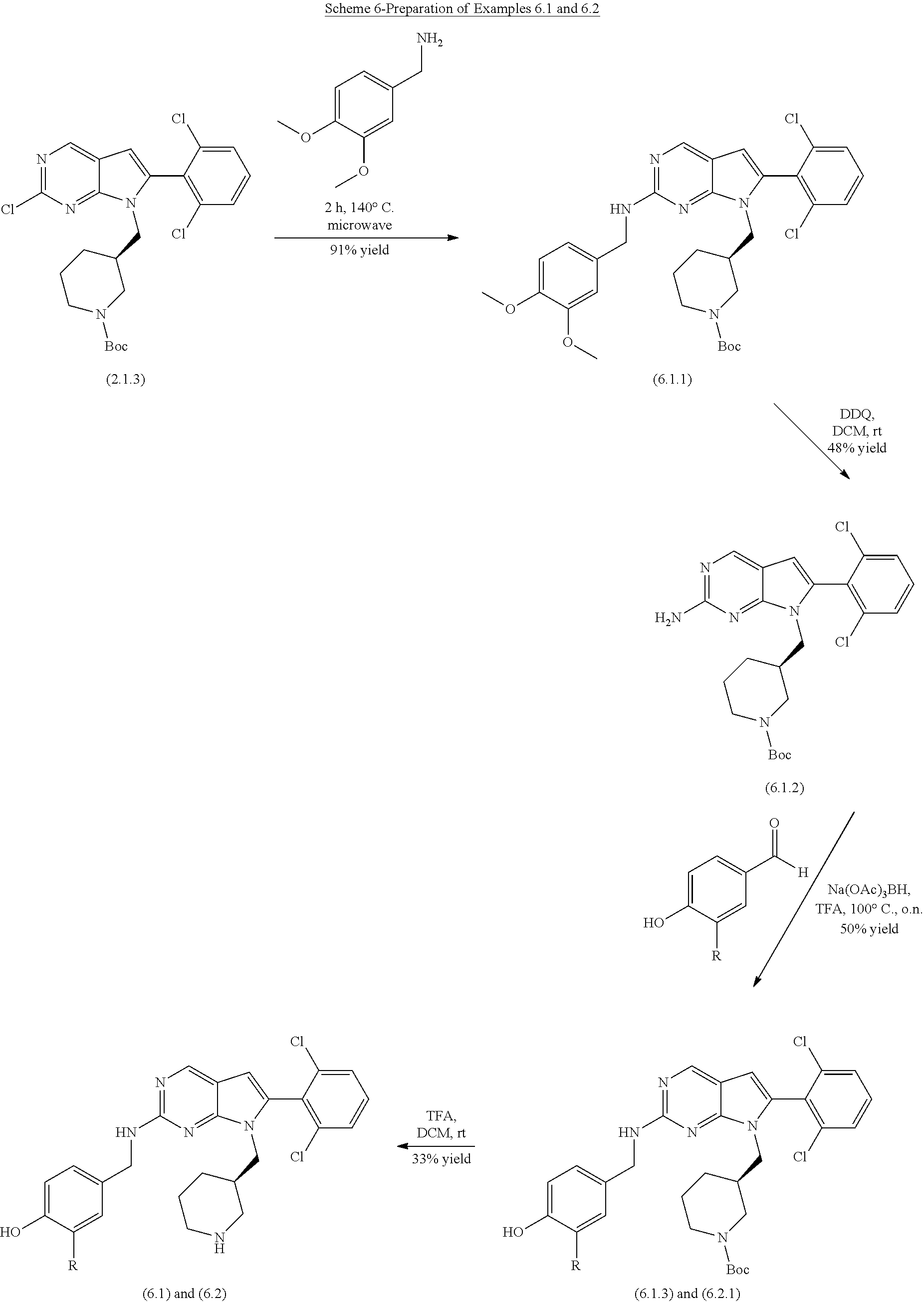

### EXAMPLE 6.1

### (R)-2-chloro-4-((6-(2,6-dichlorophenyl)-7-(piperidin-3-ylmethyl)-7H-pyrrolo[2,3-d]pyrimidin-2-ylamino)methyl)phenol

### (6.1.1) (S)-tert-butyl 3-((6-(2,6-dichlorophenyl)-2-(3,4-dimethoxybenzylamino)-7H-pyrrolo[2,3-d]pyrimidin-7-yl)methyl)piperidine-1-carboxylate

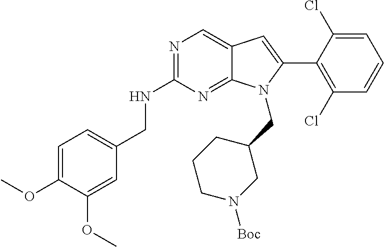

Compound (2.1.3) (110 mg, 0.22 mmol) was dissolved in 3,4-dimethoxybenzylamine (1.0 mL, 6.63 mmol) and the solution was subsequently heated in the microwave for 2 hours at 140° C. The reaction mixture was then diluted with EtOAc and washed with NH₄Cl solution (1×) and brine (1×). The organic layer was dried (Na₂SO₄), filtered and concentrated in vacuo. The crude product was purified by column chromatography (SiO₂, heptane/EtOAc; 6/4 as mobile phase). The title compound was obtained in 91% yield (126 mg, 0.20 mmol). LC-MS: peak at 3.82 min., mass [M+H]=626.

### (6.1.2) (S)-tert-butyl 3-((2-amino-6-(2,6-dichlorophenyl)-7H-pyrrolo[2,3-d]pyrimidin-7-yl)methyl)piperidine-1-carboxylate

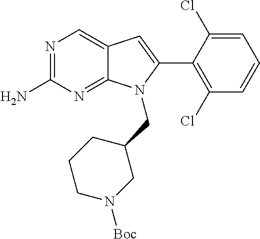

Compound (6.1.1) (126 mg, 0.20 mmol) was dissolved in DCM (1.0 mL). To the solution was added 1,2-dicarbonitrile-4,5-dichloro-3,6,dioxo-1,4-cyclohexadiene (45 mg, 0.20 mmol). The reaction mixture was stirred at room temperature for 1 hour and was then concentrated in vacuo. The crude product was purified by column chromatography (SiO₂, DCM/MeOH; 9.5:0.5 as mobile phase) and the title was obtained in 48% yield (46 mg, 0.10 mmol). LC-MS: peak at 2.67 min., mass [M+H]=476.

### (6.1.3) (S)-tert-butyl 3-((2-(3-chloro-4-hydroxybenzylamino)-6-(2,6-dichlorophenyl)-7H-pyrrolo[2,3-d]pyrimidin-7-yl)methyl)piperidine-1-carboxylate

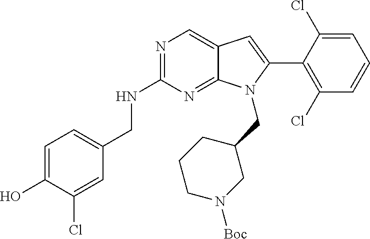

Compound (6.1.2) (46 mg, 0.10 mmol) was dissolved in DMF (1 mL) and 3-chloro-4-hydroxybenzaldehyde (30 mg, 0.20 mmol) and sodium triacetoxyborohydride (61 mg, 0.30 mmol) were added at room temperature. The reaction mixture was acidified with TFA (to pH=5), and stirred at 100° C. overnight. Next day, the reaction mixture was cooled to room temperature, diluted with EtOAc, washed with NaHCO₃ solution (1×), H₂O (3×) and brine (1×). The organic layer was dried (Na₂SO₄), filtered and concentrated in vacuo. The crude product was purified by column chromatography (SiO₂, DCM/MeOH 24:1 as mobile phase) and the title compound was obtained in 50% yield (30 mg, 0.05 mmol). LC-MS: peak at 3.85 min., mass [M+H]=616.

### (6.1) (R)-2-chloro-4-((6-(2,6-dichlorophenyl)-7-(piperidin-3-ylmethyl)-7H-pyrrolo[2,3-d]pyrimidin-2-ylamino)methyl)phenol

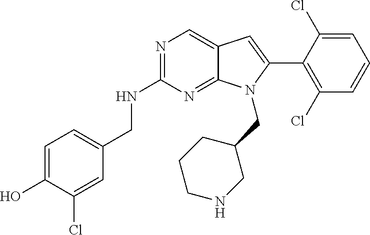

To a stirred solution of compound (6.1.3) (30 mg, 0.05 mmol) in DCM (1 mL) was added trifluoroacetic acid (0.35 mL, 4.71 mmol) at room temperature. The reaction mixture was stirred for 2 h at room temperature and was subsequently concentrated in vacuo. The crude product was purified by preparative HPLC (0-20% ACN with TFA, as mobile phase). Product fractions were combined, concentrated in vacuo and lyophilized from water/ACN. The TFA-salt of the title compound was obtained in 33% yield (10 mg, 0.02 mmol). LC-MS: peak at 2.81 min., mass [M+H]=516.

### EXAMPLE 6.2

### (R)-4-((6-(2,6-dichlorophenyl)-7-(piperidin-3-ylmethyl)-7H-pyrrolo[2,3-d]pyrimidin-2-ylamino)methyl)-2-fluorophenol

### (6.2.1) (S)-tert-butyl 3-((6-(2,6-dichlorophenyl)-2-(3-fluoro-4-hydroxybenzylamino)-7H-pyrrolo[2,3-d]pyrimidin-7-yl)methyl)piperidine-1-carboxylate

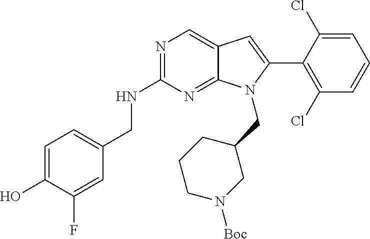

An analogous procedure to that described above to prepare compound (6.1.3) was used, except that 3-fluoro-4-hydroxybenzaldehyde was used instead of 3-chloro-4-hydroxybenzaldehyde. Yield=23% (33 mg, 0.055 mmol). MS (ESI): mass [M+H]=600.

### (6.2) (R)-4-((6-(2,6-dichlorophenyl)-7-(piperidin-3-ylmethyl)-7H-pyrrolo[2,3-d]pyrimidin-2-ylamino)methyl)-2-fluorophenol

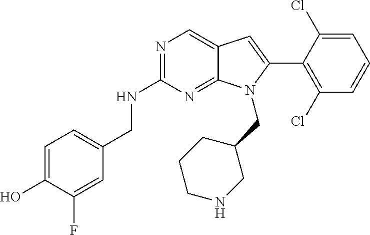

An analogous procedure to that described above to prepare compound 6.1 was used, except that that compound (6.2.1) was used instead of compound (6.1.3). Yield=53% (18 mg, 0.03 mmol). UPLC-MS: peak at 2.05 min., mass [M+H]=500.

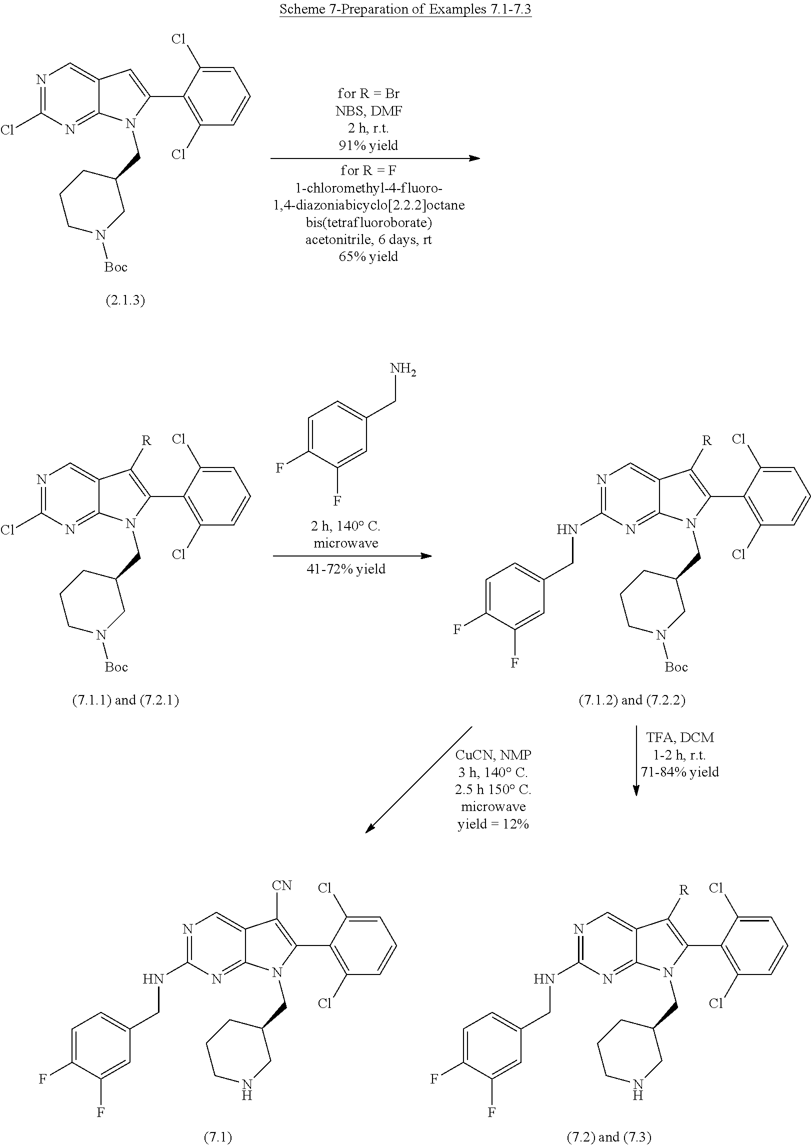

### EXAMPLE 7.1

### (R)-6-(2,6-dichlorophenyl)-2-(3,4-difluorobenzylamino)-7-(piperidin-3-ylmethyl)-7H-pyrrolo[2,3-d]pyrimidine-5-carbonitrile

### (7.1.1) (S)-tert-butyl 3-((5-bromo-2-chloro-6-(2,6-dichlorophenyl)-7H-pyrrolo[2,3-d]pyrimidin-7-yl)methyl)piperidine-1-carboxylate

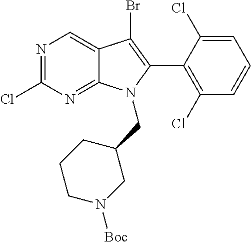

At room temperature N-bromosuccinimide (86 mg, 0.48 mmol) was added to a solution of (2.1.3) (219 mg, 0.44 mmol) in DMF (2 mL). The reaction mixture was stirred overnight and was then diluted with EtOAc and vigorously stirred with 10% Na₂S₂O₃-solution. The organic layer was separated from the aqueous layer and stirred again with 10% Na₂S₂O₃-solution. Subsequently, the organic layer was washed with NaHCO₃-solution (1×) and brine (1×). The organic layer was dried (Na₂SO₄), filtered and concentrated in vacuo. The crude product was purified by column chromatography (SiO₂, heptane/EtOAc; 100% heptane to 30% EtOAc as mobile phase) to give the title compound in 91% yield (230 mg, 0.40 mmol). LC-MS: peak at 5.20 min., mass [M+H]: 573.

### (7.1.2) (S)-tert-butyl 3-((5-bromo-6-(2,6-dichlorophenyl)-2-(3,4-difluorobenzylamino)-7H-pyrrolo[2,3-d]pyrimidin-7-yl)methyl)piperidine-1-carboxylate

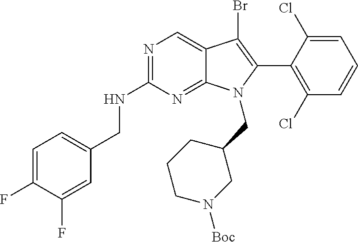

A solution of compound (7.1.1) (230 mg, 0.400 mmol) in 3,4-difluorobenzylamine (1.21 g, 1 mL, 8.45 mmol) was heated in the microwave for 2 h at 140° C. The reaction mixture was diluted with EtOAc and washed with 1M aqueous HCl-solution (2×) and brine (2×). The organic layer was dried (Na₂SO₄), filtered and concentrated in vacuo. The crude product was purified by column chromatography (SiO₂, heptane/EtOAc; 100% heptane to 40% EtOAc as mobile phase) to give the title compound in 72% yield (195 mg, 0.29 mmol). LC-MS: peak at 4.53 min., mass [M+H]: 680.

### (7.1) (R)-6-(2,6-dichlorophenyl)-2-(3,4-difluorobenzylamino)-7-(piperidin-3-ylmethyl)-7H-pyrrolo[2,3-d]pyrimidine-5-carbonitrile

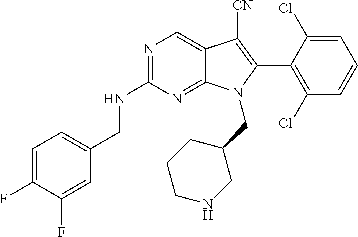

To a solution of compound (7.1.2) (50 mg, 0.073 mmol) in NMP (1.2 mL) was added copper(I) cyanide (14 mg, 0.15 mmol). The reaction mixture was heated in the microwave for 3 h at 140° C., followed by heating for 2.5 h at 150° C. Subsequently, the reaction mixture was diluted with DCM/MeOH (9/1) and washed with aqueous NH₄OH solution (1×), brine (2×), dried (Na₂SO₄) and concentrated in vacuo. The crude product was purified with preparative HPLC (0-50% ACN with TFA, as mobile phase). Product fractions were neutralized with aqueous NaHCO₃-solution, extracted once with DCM/MeOH (9/1), washed with brine (1×), dried (Na₂SO₄), concentrated in vacuo, dissolved in a mixture of EtOH/water and finally lyophilized to obtain the title compound in 12% yield (5 mg, 0.009 mmol). LC-MS: peak at 3.46 min., mass [M+H]: 527.

### EXAMPLE 7.2

### (R)-6-(2,6-dichlorophenyl)-N-(3,4-difluorobenzyl)-5-fluoro-7-(piperidin-3-ylmethyl)-7H-pyrrolo[2,3-d]pyrimidin-2-amine

### (7.2.1) (S)-tert-butyl 3-((2-chloro-6-(2,6-dichlorophenyl)-5-fluoro-7H-pyrrolo[2,3-d]pyrimidin-7-yl)methyl)piperidine-1-carboxylate

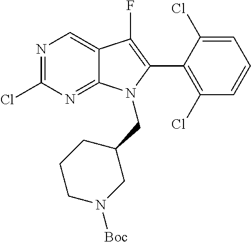

To a solution of compound (2.1.3) (59 mg, 0.119 mmol) in acetonitrile (1 mL) was added 1-chloromethyl-4-fluoro-1,4-diazoniabicyclo[2.2.2]octane bis(tetrafluoroborate) (55 mg, 0.155 mmol) at room temperature and the reaction mixture was stirred for 6 days. The reaction mixture was diluted with EtOAc and washed with water (2×) and brine (1×). The organic layer was dried (Na₂SO₄), filtered and concentrated in vacuo to give crude product. The crude product was purified by column chromatography (SiO₂, heptane/EtOAc; 100% heptane to 30% EtOAc as mobile phase) to give the title compound in 65% yield (40 mg, 0.078 mmol). LC-MS: peak at 5.05 min., mass [M+H]: 513.

### (7.2.2) (S)-tert-butyl 3-((6-(2,6-dichlorophenyl)-2-(3,4-difluorobenzylamino)-5-fluoro-7H-pyrrolo[2,3-d]pyrimidin-7-yl)methyl)piperidine-1-carboxylate

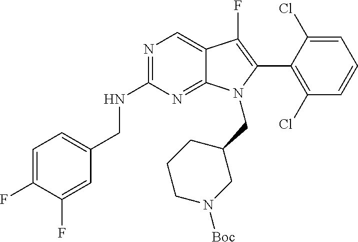

An analogous procedure to that described above to prepare compound (7.1.2) was used, except that compound (7.2.1) was used instead of compound (7.1.1). Yield=41% (20 mg, 0.032 mmol). LC-MS: peak at 4.43 min., mass [M+H]: 620.

### (7.2) (R)-6-(2,6-dichlorophenyl)-N-(3,4-difluorobenzyl)-5-fluoro-7-(piperidin-3-ylmethyl)-7H-pyrrolo[2,3-d]pyrimidin-2-amine

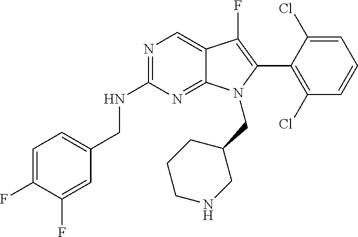

To a solution of compound (7.2.2) (20 mg, 0.032 mmol) in DCM (2 mL) was added TFA (1 mL, 13.46 mmol) at room temperature and the reaction mixture was stirred for 2 h. Then, the reaction mixture was concentrated in vacuo and the crude product was purified with preparative HPLC (0-50% ACN with TFA, as mobile phase). After lyophilization from ACN/water the title compound was obtained as a TFA-salt in 71% yield (14.6 mg, 0.023 mmol). UPLC-MS: peak at 2.64 min., mass [M+H]=520.

### EXAMPLE 7.3

### (R)-5-bromo-6-(2,6-dichlorophenyl)-N-(3,4-difluorobenzyl)-7-(piperidin-3-ylmethyl)-7H-pyrrolo[2,3-d]pyrimidin-2-amine

### (7.3) (R)-5-bromo-6-(2,6-dichlorophenyl)-N-(3,4-difluorobenzyl)-7-(piperidin-3-ylmethyl)-7H-pyrrolo[2,3-d]pyrimidin-2-amine

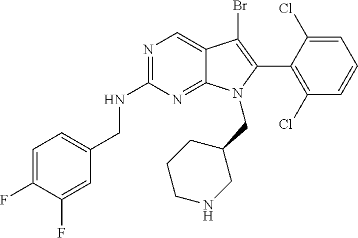

To a solution of compound (7.1.2) (36 mg, 0.053 mmol) in DCM (2 mL) was added TFA (0.29 mL, 3.96 mmol) at room temperature and the reaction mixture was stirred for 1 h. The reaction mixture was concentrated in vacuo and the crude product was dissolved in DCM, followed by neutralization with aqueous NaHCO₃-solution. The organic layer was separated from the aqueous layer by means of a DCM/water-separation filter and concentrated in vacuo. The product was lyophilized from ACN/water to give the title compound in 84% yield (26 mg, 0.045 mmol). UPLC-MS: peak at 0.92 min., mass [M+H]=580.

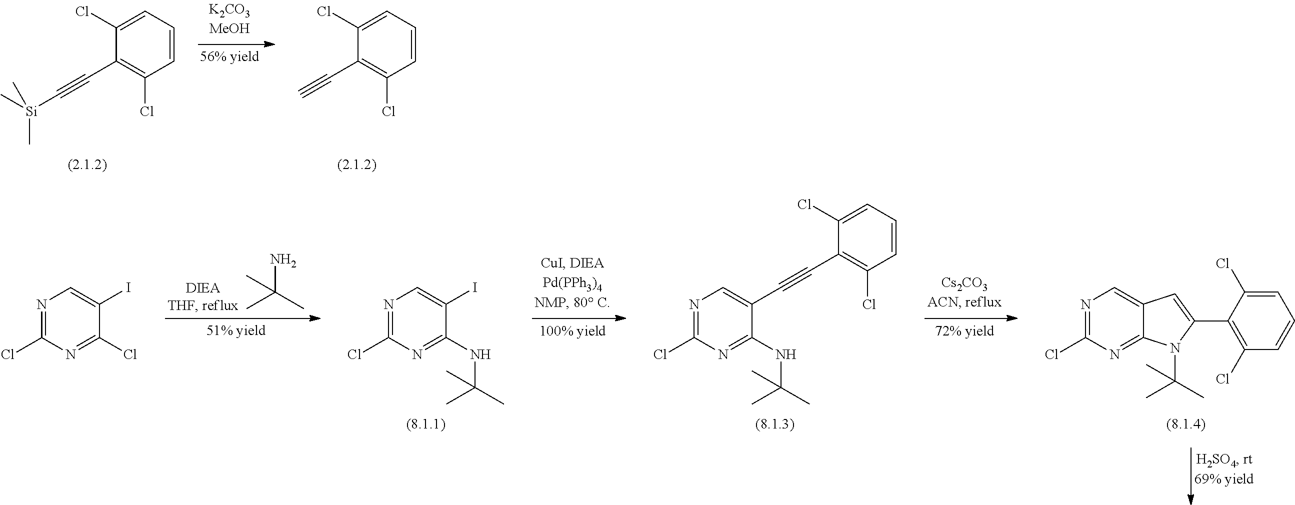

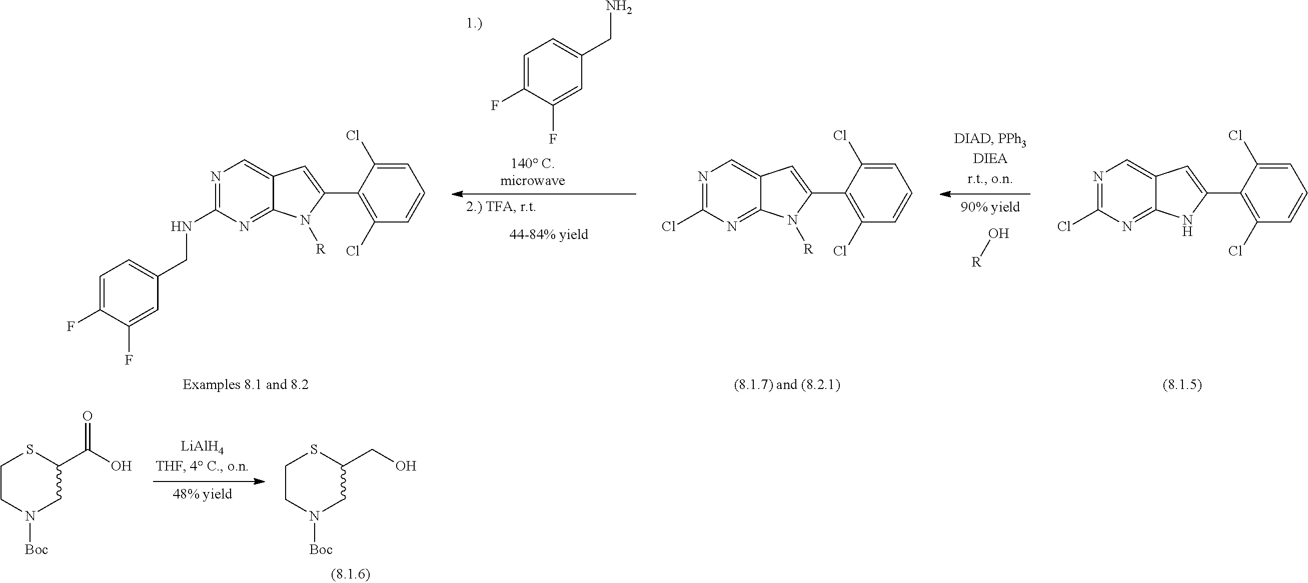

### EXAMPLE 8.1

### 6-(2,6-Dichlorophenyl)-N-(3,4-difluorobenzyl)-7-(thiomorpholin-2-yl-methyl)-7H-pyrrolo[2,3-d]pyrimidin-2-amine

### (8.1.1) N-tert-butyl-2-chloro-5-iodopyrimidin-4-amine

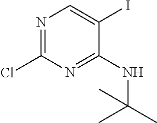

To a solution of 2,4-dichloro-5-iodopyrimidine (10 g, 36.4 mmol) in THF (150 mL) and DIEA (5.17 g, 6.97 mL, 40.0 mmol) was added dropwise a solution of tert-butylamine (2.85 g, 4.09 mL, 38.9 mmol) in THF (15 mL). The reaction was heated to reflux and stirred over weekend at reflux temperature. The reaction mixture was cooled to room temperature, diluted with EtOAc and washed with Na₂CO₃-solution (2×) and brine (1×). The organic layer was dried (Na₂SO₄) and concentrated in vacuo to give crude product, which was purified by column chromatography (SiO₂, heptane/EtOAc; 100% heptane to 20% EtOAc as mobile phase) to give the title compound in 52% yield (5.84 g, 18.7 mmol).

### (8.1.2) 1,3-Dichloro-2-ethynylbenzene

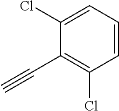

To a solution of compound (2.1.2) (9.28 g, 38.2 mmol) in methanol (60 mL) was added potassium carbonate (0.74 g, 5.34 mmol) and the reaction mixture was stirred overnight at room temperature. The reaction mixture was concentrated in vacuo to give crude product. This was dissolved in DCM and the organic layer was washed with saturated NaHCO₃-solution and then concentrated in vacuo. The residu was crystallized from heptane/DCM 9:1 and the crystals were filtered to give the title compound in 57% yield (3.7 g, 21.63 mmol).

### (8.1.3) N-tert-Butyl-2-chloro-5-((2,6-dichlorophenyl)ethynyl)pyrimidin-4-amine

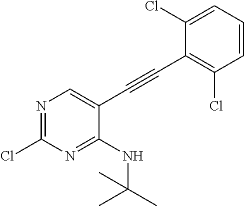

A nitrogen flow was bubbled through a solution of compound (8.1.1) (2.06 g, 6.61 mmol) in NMP (45 mL) for 10 minutes. Subsequently, compound (8.1.2) (1.36 g, 7.93 mmol), copper(I) iodide (0.031 g 0.165 mmol) and then DIEA (1.28 g, 1.64 mL, 9.92 mmol) and Pd(PPh₃)₄ (0.38 g, 0.331 mmol) were added at room temperature. The reaction mixture was heated to 80° C. for 2 h and then cooled to room temperature. Water was added and the reaction mixture was stirred overnight at room temperature. The reaction mixture was taken into a lot of water and extracted with EtOAc. The organic layer was washed with water (2×), brine (1×); aqueous layers were combined and extracted with EtOAc (2×). All organic layers were combined, dried (Na₂SO₄) and evaporated till dryness. Crude product was taken into DCM and purified by column chromatography (SiO₂, heptane/EtOAc; 100% heptane to 20% EtOAc as mobile phase) to give the title compound in quantitative yield (2.5 g, 7.05 mmol). LC-MS: peak at 5.57 min., mass [M+H]: 354.

### (8.1.4) 7-tert-Butyl-2-chloro-6-(2,6-dichlorophenyl)-7H-pyrrolo[2,3-d]pyrimidine

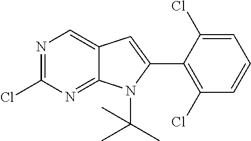

To a stirred suspension of compound (8.1.3) (2.5 g, 7.05 mmol) in acetonitrile (200 mL) was added cesium carbonate (5.05 g, 15.51 mmol) and the reaction mixture was stirred at reflux temperature overnight. The reaction mixture was cooled to room temperature and taken into EtOAc/water. The layers were separated and the organic layer was washed with brine (1×), dried (Na₂SO₄) and evaporated till dryness. The crude product was purified by column chromatography (SiO₂, heptane/EtOAc; 100% heptane to 20% EtOAc as mobile phase) to give the title compound in 72% yield (1.8 g, 5.08 mmol). LC-MS: peak at 4.97 min., mass [M+H]: 354.

### (8.1.5) 2-Chloro-6-(2,6-dichlorophenyl)-7H-pyrrolo[2,3-d]pyrimidine

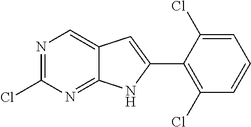

Compound (8.1.4) (2.44 g, 6.88 mmol) was dissolved in concentrated H₂SO₄ (10 mL) and stirred at room temperature overnight. Subsequently, the reaction mixture was quenched carefully in saturated NaHCO₃-solution and then extracted with EtOAc. The organic layer was washed with brine (1×), dried (Na₂SO₄) and evaporated till dryness. The crude product was taken into DCM/MeOH 9/1 and purified by column chromatography (SiO₂, heptane/EtOAc; 100% heptane to 50% EtOAc as mobile phase) to give the title compound in 69% yield (1.43 g, 4.77 mmol). LC-MS: peak at 4.12 min., mass [M+H]: 298.

### (8.1.6) tert-Butyl 2-(hydroxymethyl)thiomorpholine-4-carboxylate

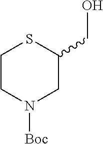

Thiomorpholine-2,4-dicarboxylic acid 4-tert-butyl ester (0.20 g, 0.809 mmol) was suspended in THF (15 mL) and cooled to 4° C. Then, lithium aluminum hydride (92 mg, 2.43 mmol) was added in small portions. The reaction mixture was allowed to warm up to room temperature overnight. Next day, The reaction was quenched by addition of saturated Na₂SO₄ solution (2 mL) and EtOAc (22 mL). The reaction mixture was stirred overnight and filtered over dicalite. The filtrate was evaporated till dryness and the crude product was purified by column chromatography (SiO₂, heptane/EtOAc; 100% heptane to 40% EtOAc as mobile phase) to give the title compound in 48% yield (91 mg, 0.39 mmol).

### (8.1.7) tert-Butyl 2-((2-chloro-6-(2,6-dichlorophenyl)-7H-pyrrolo[2,3-d]pyrimidin-7-yl)methyl)thiomorpholine-4-carboxylate

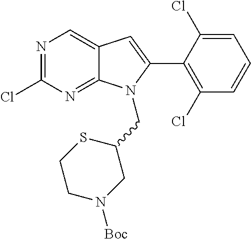

To a solution of compound (8.1.5) (65 mg, 0.218 mmol) and compound (8.1.6) (91 mg, 0.390 mmol) in THF (8 mL) was added DIEA (84 mg, 0.114 mL, 0.653 mmol), PPh₃ (143 mg, 0.544 mmol) and DIAD (0.544 mmol, 0.108 ml, 110 mg) at room temperature. The reaction mixture was stirred overnight at room temperature. Next day, EtOAc was added to the reaction mixture, which was subsequently extracted with water (2×), brine (1×), dried (Na₂SO₄) and evaporated till dryness. The crude product was purified by column chromatography (SiO₂, heptane/EtOAc; 100% heptane to 50% EtOAc as mobile phase) to give the title compound in 90% yield (101 mg, 0.197 mmol). LC-MS: peak at 4.80 min., mass [M+H]: 513.

### (8.1) 6-(2,6-Dichlorophenyl)-N-(3,4-difluorobenzyl)-7-(thiomorpholin-2-ylmethyl)-7H-pyrrolo[2,3-d]pyrimidin-2-amine

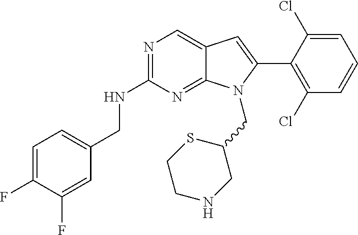

A solution of compound (8.1.6) (101 mg, 0.197 mmol) in 3,4-difluorobenzylamine (2 mL, 2.42 g, 16.91 mmol) was heated in the microwave at 140° C. for 2 h. Subsequently, the reaction mixture was taken into EtOAc and extracted with saturated NH₄Cl-solution (1×), water (2×) and brine (1×); The aqueous layers were combined and extracted again with EtOAc (1×). The organic layers were combined, dried (Na₂SO₄) and evaporated till dryness. The crude product was purified with by column chromatography (SiO₂, heptane/EtOAc; 100% heptane to 50% EtOAc as mobile phase) giving 105 mg of desired intermediate product (86% yield). This product was dissolved in DCM (3 mL) and at room temperature TFA (0.5 mL, 768 mg, 6.73 mmol) was added. The reaction mixture was stirred at room temperature overnight and next day, the reaction mixture was taken into DCM and washed with saturated NaHCO₃-solution. The organic layer was separated from the aqueous layer by means of a DCM/water-separation filter and concentrated in vacuo. The crude product was purified with preparative HPLC (0-50% ACN with TFA, as mobile phase). Pure fractions were taken into DCM and washed with saturated NaHCO₃-solution. The organic layer was separated from the aqueous layer by means of a DCM/water-separation filter and concentrated in vacuo. The product was dissolved in dioxane/water and lyophilized to yield the title compound in 98% yield (86 mg, 0.165 mmol). UPLC-MS: peak at 2.25 min., mass [M+H]=520.

### EXAMPLE 8.2

### 7-(Azepan-4-yl)-6-(2,6-dichlorophenyl)-N-(3,4-difluorobenzyl)-7H-pyrrolo[2,3-d]pyrimidin-2-amine

### (8.2.1) tert-Butyl 4-(2-chloro-6-(2,6-dichlorophenyl)-7H-pyrrolo[2,3-d]pyrimidin-7-yl)azepane-1-carboxylate

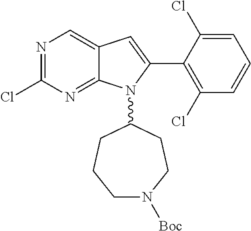

An analogous procedure to that described above to prepare compound (8.1.7) was used, except that that 4-hydroxyazepane-1-carboxylic acid tert-butyl ester was used instead of compound (8.1.6). LC-MS: peak at 3.61 min., mass [M+H]: 495.

### (8.2) 7-(Azepan-4-yl)-6-(2,6-dichlorophenyl)-N-(3,4-difluorobenzyl)-7H-pyrrolo[2,3-d]pyrimidin-2-amine

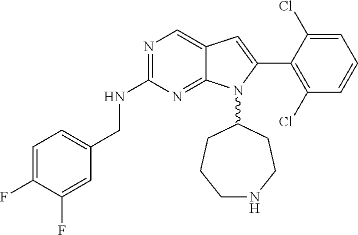

An analogous procedure to that described above to prepare compound 8.1 was used, except that that compound (8.2.1) was used instead of compound (8.1.7). Yield is 44% (19 mg, 0.38 mmol). UPLC-MS: peak at 2.46 min., mass [M+H]=502.

### EXAMPLE 9

### Assay Method

Inhibition of PKC theta kinase activity was measured using an Immobilized Metal for Phosphochemicals—based coupled assay (IMAP). IMAP is a homogeneous fluorescence polarization (FP) assay based on affinity capture of phosphorylated peptide substrates. IMAP uses fluorescein-labeled peptide substrates that, upon phosphorylation by a protein kinase, bind to so called IMAP nanoparticles, which are derivatized with trivalent metal complexes. Such binding causes a change in the rate of the molecular motion of the peptide, and results in an increase in the FP value observed for the fluorescein label attached to the substrate peptide. In this assay, PKC theta directly phosphorylates the fluorescein-labeled peptide substrate.

Enzymes, substrate and ATP are diluted at all steps in Kinase Reaction buffer (10 mM Tris-HCl, 10 mM MgCl2, 0.01% Tween-20, 0.05% NaN3 pH 7.2, 1 mM DTT). The final volume at the kinase reaction step of the assay in the 384-well plate is 20 μl. The concentrations presented within brackets are final concentrations. First, compounds or DMSO (1%) are added to the wells. Thereafter, peptide substrate (Pseudosubstrate LHQRRGSIKQAKVHHVK-FL, Neosystem, 50 nM) and ATP (10 μM) are added followed by the addition of the PKC theta enzyme (His-tagged human recombinant active PKC theta, 82 kDa, in-house purified, 10 ng/ml), and the mix is incubated for 60 minutes at 30° C. in the dark. Then IMAP progressive binding buffer (100% 1× buffer A, 1:400 Progressive Binding Reagent; Molecular Devices) is added followed by an incubation step of 60 minutes at room temperature in the dark. Finally, the FP signal is read.

TABLE 1
PKCθ activity for Compounds According to The Invention

| Example | | |
|---|---|---|
| number | Structure | IC₅₀ IMAP PKCθ* |
| (1.1) | 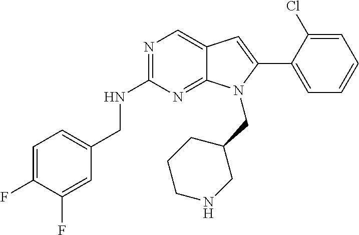 | A |
| (1.2) | 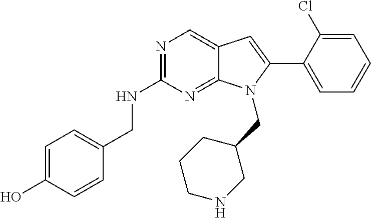 | B |
| (1.3) | 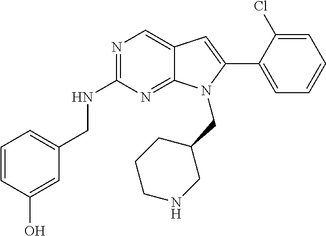 | B |
| (1.4) | 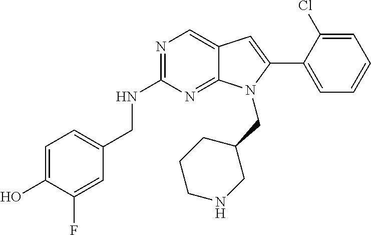 | A |
| (2.1) | 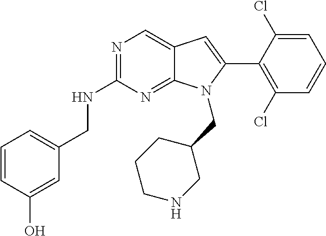 | A |
| (2.2) | 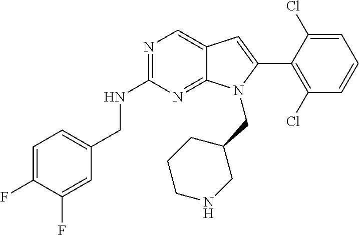 | A |
| (2.3) | 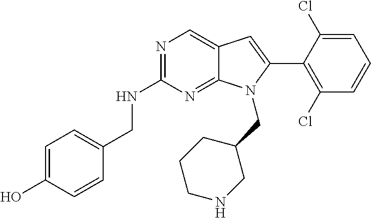 | C |
| (2.4) | 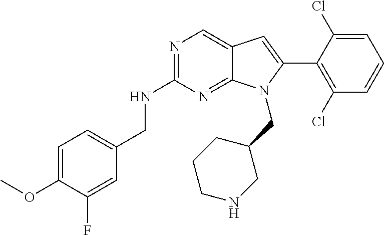 | A |
| (2.5) | 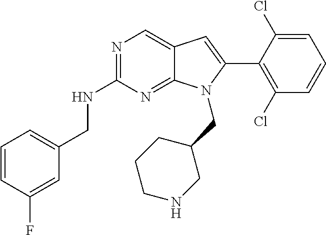 | A |
| (2.6) | 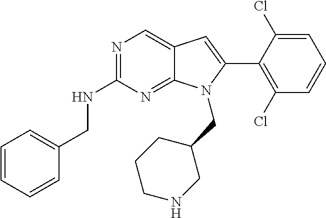 | A |
| (3.1) | 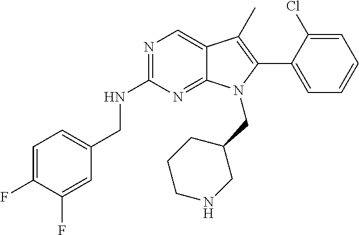 | B |
| (3.2) | 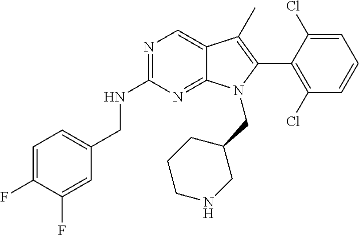 | A |
| (3.3) | 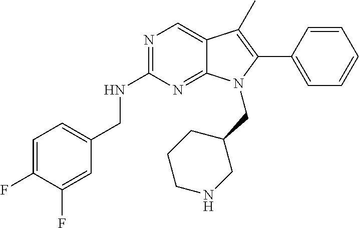 | C |
| (4.1) | 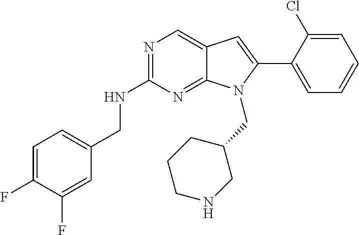 | B |
| (4.2) | 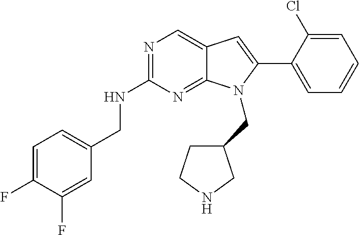 | C |
| (4.3) | 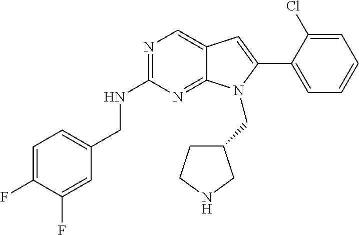 | B |
| (4.4) | 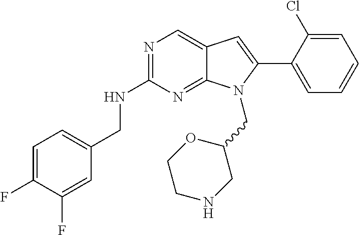 | B |
| (5.1) | 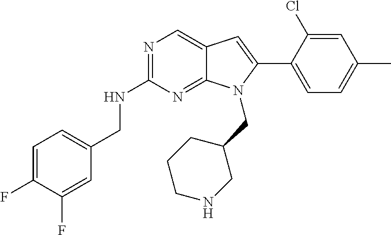 | A |
| (5.4) | 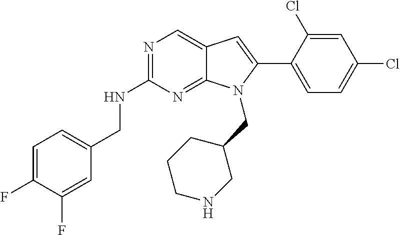 | B |
| (5.2) | 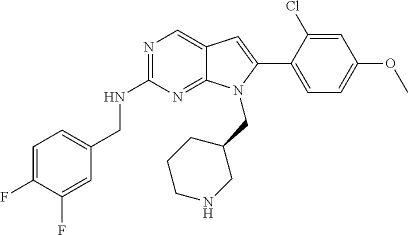 | B |
| (5.3) | 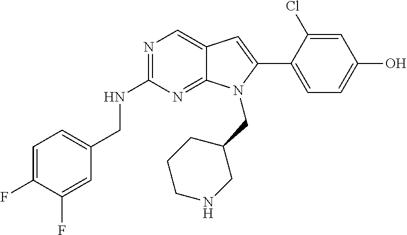 | A |
| (5.4) | 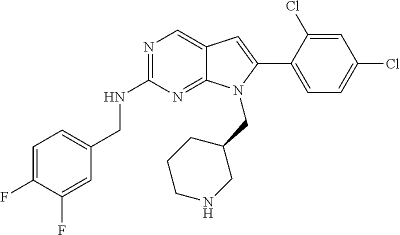 | B |
| (5.5) | 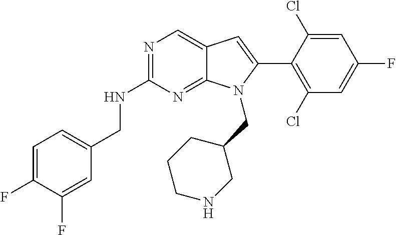 | A |
| (6.1) | 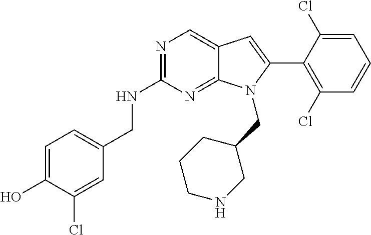 | A |
| (6.2) | 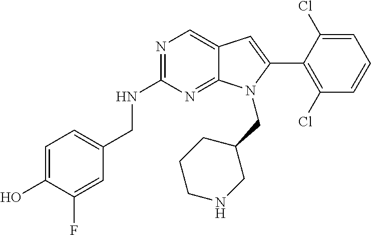 | A |
| (7.1) | 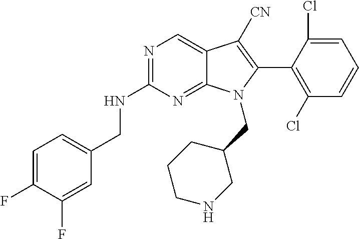 | A |
| (7.2) | 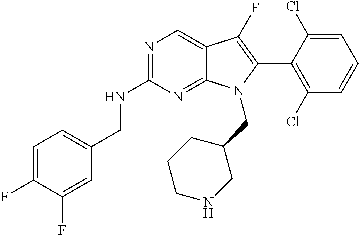 | A |
| (7.3) | 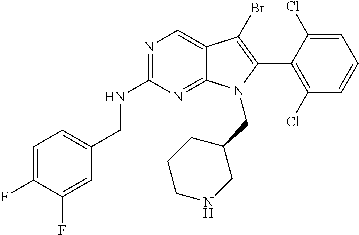 | A |
| (8.1) | 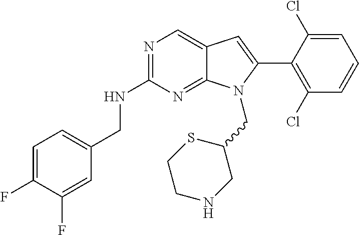 | A |
| (8.2) | 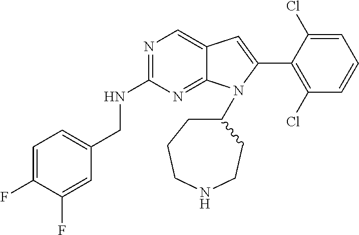 | A |

*A = IC₅₀ <10 nM, B = IC₅₀ 10-100 nM, C = IC₅₀ 100 nM - 10 μM

1. A pyrrolo[2,3-d]pyrimidin-2-yl derivative according to formula I 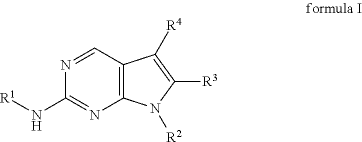
wherein
R¹ is C₆₋₁₀aryl optionally substituted with one or more substituents independently selected from halogen, hydroxy, cyano, C₁₋₆alkyl, C₃₋₆cycloalkyl, C₁₋₆alkyloxy and C₃₋₆cycloalkyloxy, said C₁₋₆alkyl, C₃₋₆ cycloalkyl, C₁₋₆alkyloxy and C₃₋₆cycloalkyloxy being optionally substituted with one or more halogens or
R¹ is C₃₋₈cycloalkyl or
R¹ is —C₁₋₃alkyl-Z, wherein Z is C₃₋₈cycloalkyl, C₆₋₁₂aryl or a 5-10 membered heteroaryl ring system comprising 1-2 heteroatoms independently selected from O, S and N, said C₆₋₁₀aryl and 5-10 membered heteroaryl ring system being optionally substituted with one or more substituents independently selected from halogen, hydroxy, cyano, C₁₋₆alkyl, C₃₋₆cycloalkyl, C₁₋₆alkyloxy and C₃₋₆cycloalkyloxy, said C₁₋₆alkyl, C₃₋₆cycloalkyl, C₁₋₆alkyloxy and C₃₋₆cycloalkyloxy being optionally substituted with one or more halogens;
R² is —C₂₋₇alkyl-NR⁵R⁶ or
R² is —C₀₋₄alkyl-Y wherein Y is a 4-8 membered saturated or unsaturated heterocyclic ring system comprising one or two heteroatomic moieties independently selected from O, S and N(R⁷)ₚ, said heterocyclic ring system being optionally substituted with halogen, hydroxy, C₁₋₆alkyl or C₁₋₆ alkyloxy or
R² is —C₀₋₂alkylC₃₋₆cycloalkyl substituted with —NR⁸R⁹ or —CH₂NR⁸R⁹;
R³ is C₁₋₆alkyl, C₆₋₁₀aryl or C₆₋₁₀arylC₁₋₃alkyl, said C₆₋₁₀aryl and C₆₋₁₀arylC₁₋₃alkyl being optionally substituted with one or more substituents independently selected from halogen, hydroxy, cyano, C₁₋₆alkyl, C₃₋₆cycloalkyl, C₁₋₆alkyloxy, C₃₋₆cycloalkyloxy, —NHCOR¹⁰, —NHS(O)_{q}R¹¹, —CONR¹²R¹³, —S(O)ᵣR¹⁴R¹⁵, and —NHCONR¹⁶R¹⁷ said C₁₋₆alkyl C₃₋₆ cycloalkyl, C₁₋₆alkyloxy and C₃₋₆cycloalkyloxy being optionally substituted with one or more halogens;
R⁴ is H, C₁₋₆alkyl, CN or halogen;
R⁵-R⁹ are independently chosen from H and C₁₋₄alkyl;
R¹⁰ and R¹¹ are independently C₁₋₄alkyl;
R¹² and R¹³ are independently chosen from H and C₁₋₄alkyl;
R¹⁴-R¹⁷ are independently C₁₋₄alkyl;
p is 0 or 1 and
q and r are independently 1 or 2
or a pharmaceutically acceptable salt or solvate thereof.

2. The pyrrolo[2,3-d]pyrimidin-2-yl-amine derivative according to claim 1, wherein R¹ is —CH₂Z and wherein Z is phenyl optionally substituted with one or more substituents independently selected from halogen, hydroxyl, cyano C₁₋₆alkyl, C₃₋₆cycloalkyl, C₁₋₆alkyloxy and C₃₋₆cycloalkyloxy said C₁₋₆alkyl, C₃₋₆cycloalkyl, C₁₋₆alkyloxy and C₃₋₆cycloalkyloxy being optionally substituted with one or more halogens;

3. The 2-pyrrolo[2,3-d]pyrimidin-2-yl-amine derivative according to claim 2, wherein R¹ is —CH₂Z and whererin Z is phenyl optionally substituted with one or more substituents independently selected from chloro, bromo, fluoro, methyl, hydroxy and methoxy;

4. The pyrrolo[2,3-d]pyrimidin-2-yl-amine derivative according to claim 1, wherein R¹ is —CH₂Z and wherein Z is a 5-10 membered heteroaryl ring system comprising 1-2 heteroatoms independently selected from O, S and N and being optionally substituted with one or more substituents independently selected from chloro, fluoro, bromo, methyl, hydroxy and methoxy.

5. The pyrrolo[2,3-d]pyrimidin-2-yl-amine derivative according to claim 1, wherein R² is —CH₂Y and wherein Y is is a 4-8 membered saturated or unsaturated heterocyclic ring system comprising one or two heteroatomic moieties independently selected from O, S and N(R⁷)ₚ, said heterocyclic ring system being optionally substituted with halogen, hydroxy, C₁₋₆alkyl or C₁₋₆ alkyloxy.

6. The pyrrolo[2,3-d]pyrimidin-2-yl-amine derivative according to claim 5, wherein R² is —CH₂Y and wherein Y is piperidinyl, morpholinyl or pyrrolidinyl.

7. The pyrrolo[2,3-d]pyrimidin-2-yl derivative according to claim 1, wherein R³ is C₆₋₁₀aryl optionally substituted with one or more substituents independently selected from halogen, hydroxy, cyano, C₁₋₆alkyl, C₃₋₆cycloalkyl and C₁₋₆alkyloxy, said C₁₋₆alkyl, C₃₋₆ cycloalkyl and C₁₋₆alkyloxy being optionally substituted with one or more halogens.

8. The pyrrolo[2,3-d]pyrimidin-2-yl-amine derivative according to claim 7, wherein R³ is C₆₋₁₀aryl optionally substituted with one or more substituents independently selected from chloro, fluoro, methyl, hydroxy and methoxy;

9. The pyrrolo[2,3-d]pyrimidin-2-yl-amine derivative according to claim 1, wherein R⁴ is H, methyl, fluoro, chloro, bromo or nitrile.

10. A pyrrolo[2,3-d]pyrimidin-2-yl-amine derivative selected from 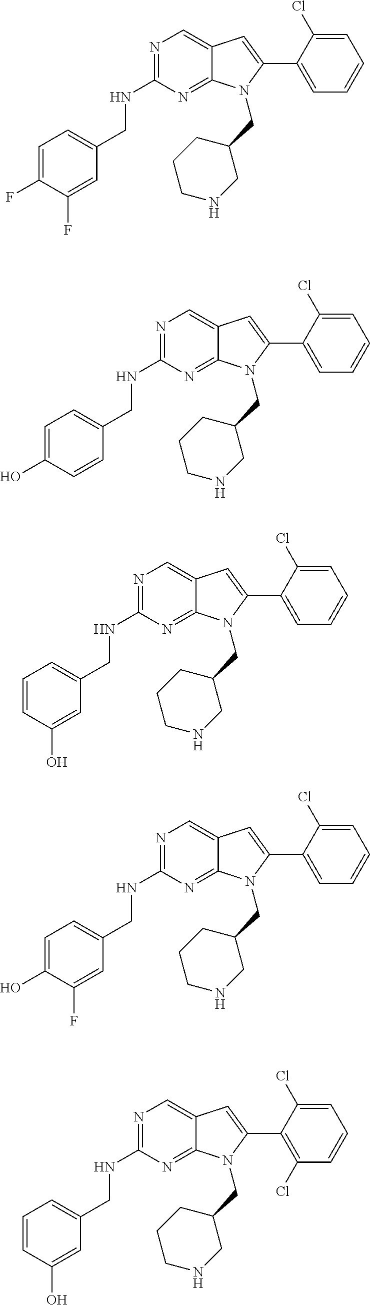 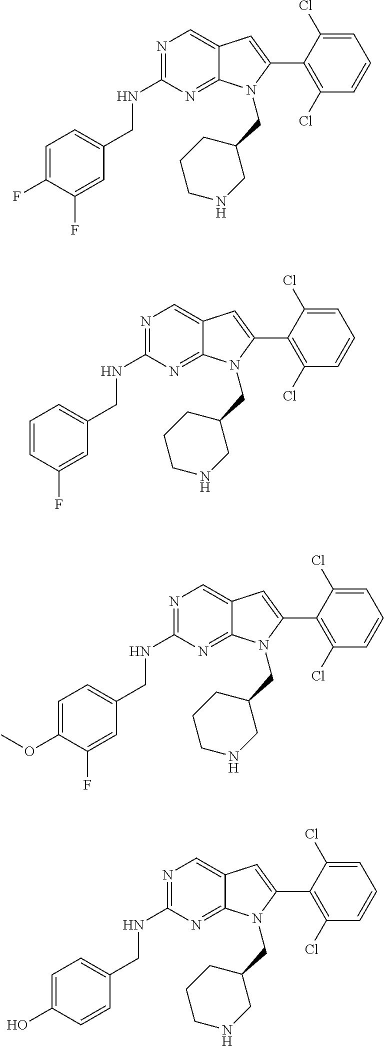 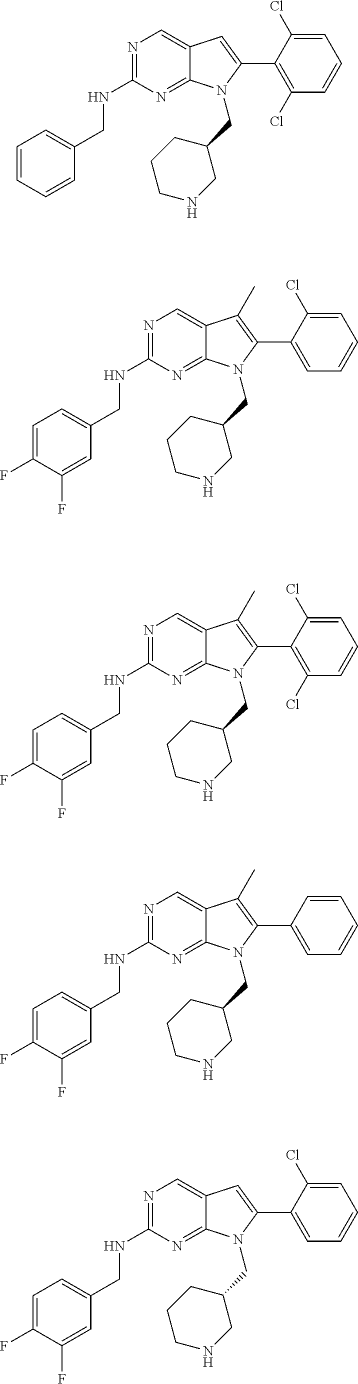 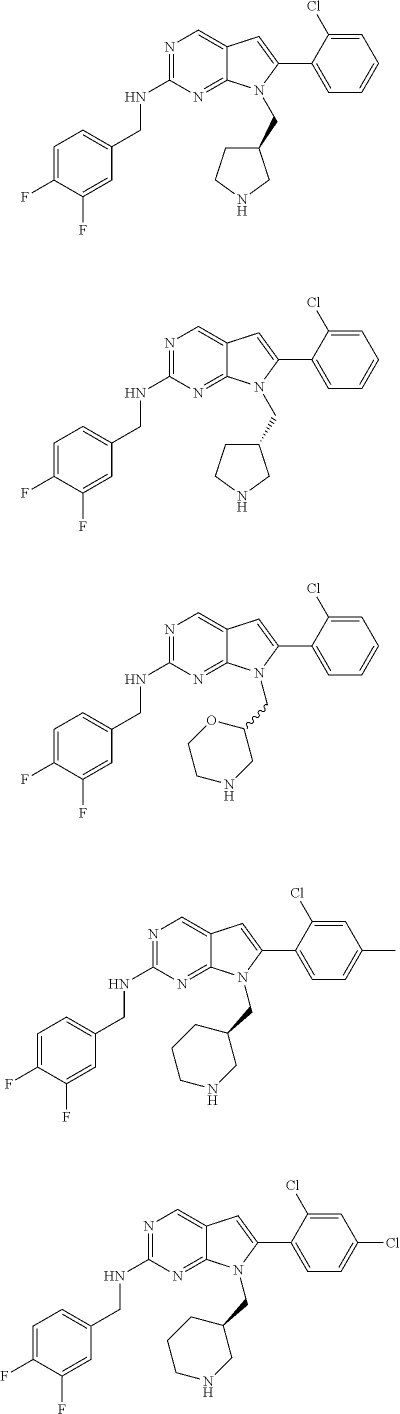 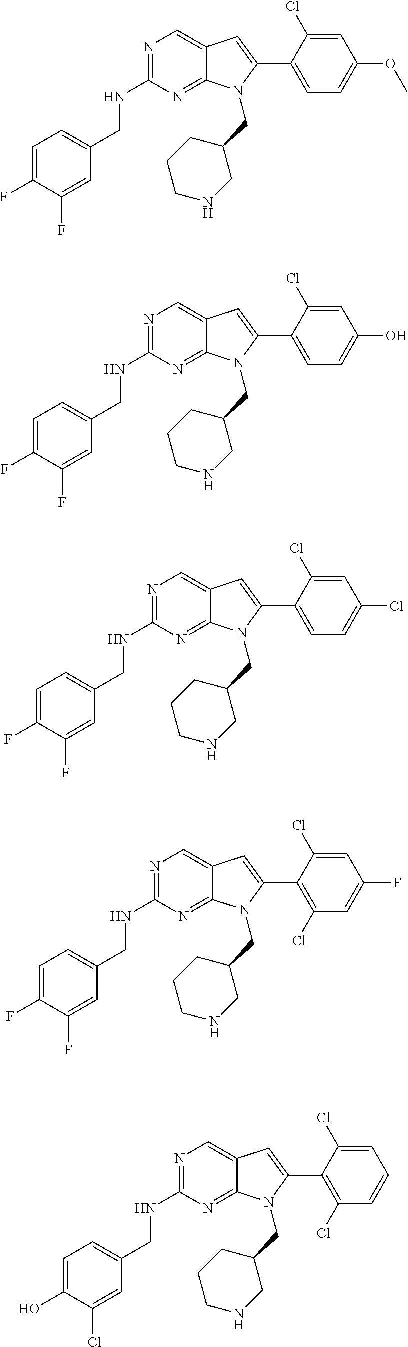 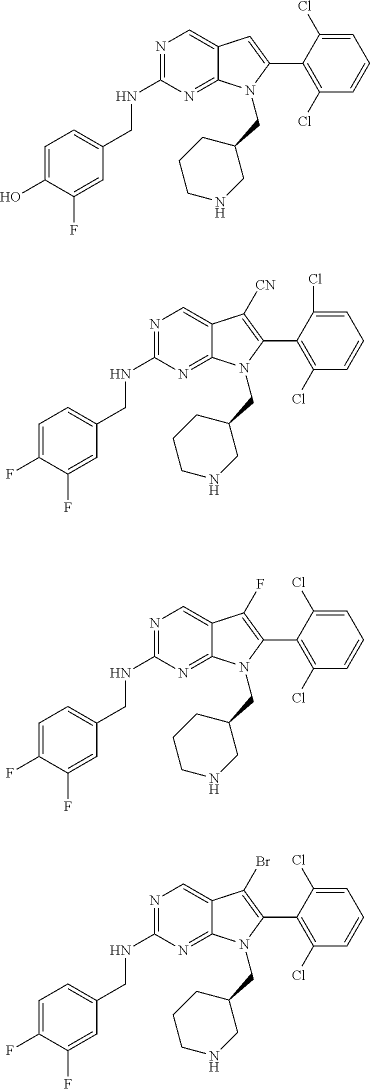 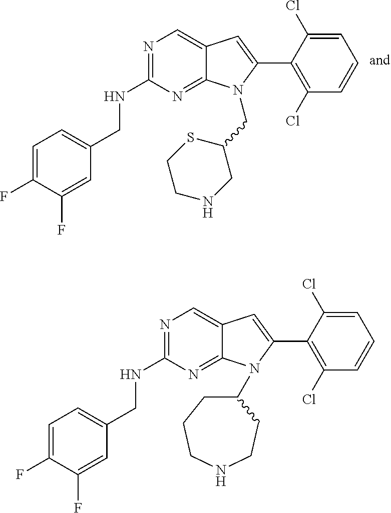
or a pharmaceutically acceptable salt or solvate thereof.

11. The pyrrolo[2,3-d]pyrimidin-2-yl-amine derivative according to claim 1 for use in therapy.

12. A pharmaceutical composition comprising a pyrrolo[2,3-d]pyrimidin-2-yl-amine derivative according to claim 1 in admixture with one or more pharmaceutically acceptable auxiliary.

13. The pyrrolo[2,3-d]pyrimidin-2-yl-amine derivative according to claim 1 for use in the treatment of PKCθ mediated disorders.

14. The pyrrolo[2,3-d]pyrimidin-2-yl-amine according to claim 13, for use in the treatment of an autoimmune or an inflammatory disease.

15. The pyrrolo[2,3-d]pyrimidin-2-yl-amine derivative according to claim 10 for use in therapy.

16. A pharmaceutical composition comprising a pyrrolo[2,3-d]pyrimidin-2-yl-amine derivative according to claim 10 in admixture with one or more pharmaceutically acceptable auxiliary.

17. The pyrrolo[2,3-d]pyrimidin-2-yl-amine derivative according to claim 10 for use in the treatment of PKCθ mediated disorders.

18. The pyrrolo[2,3-d]pyrimidin-2-yl-amine according to claim 17, for use in the treatment of an autoimmune or an inflammatory disease.
